SEQUENCE LISTING (2 sequences)

SEQ ID NO: 1 - DNA, 31 bp, Artificial Sequence
  PCR Primer
gagaagaattcagtggcaatgagagtgaagg

SEQ ID NO: 2 - DNA, 47 bp, Artificial Sequence
  PCR Primer
cctgtcggctagcctcgatcatgggaggagggtctgaaacgataatg

(19) United States
(12) Patent Application Publication
(10) Pub. No.: US 2005/0090428 A1
(43) Pub. Date: Apr. 28, 2005

(54) Porphyrins with virucidal activity

(75) Inventors: Richard W. Compans, Atlanta, GA (US); Luigi G. Marzilli, Baton Rouge, LA (US); Amy E. Sears, St. Petersburg, FL (US); Dabbey W. Dixon, Atlanta, GA (US)

Correspondence Address: PATREA L. PABST;PABST PATENT GROUP LLP, 400 COLONY SQUARE, SUITE 1200, ATLANTA, GA, 30361, US

(73) Assignees: Emory University, Atlanta, GA (US); Georgia State University Research Foundation, Inc, Atlanta, GA (US)

(21) Appl. No.: 10/500,884

(22) PCT Filed: Jan. 8, 2003
(86) PCT No.: PCT/US03/00532

Related U.S. Application Data
(60) Provisional application No. 60/347,197, filed on Jan. 8, 2002.

(51) Int. Cl. A61K 38/16; A61K 31/675; A61K 31/555; A61K 31/4439; A61K 31/4178; A61K 31/409

(52) U.S. Cl. 514/6; 514/185; 514/81; 514/338; 514/397; 514/410

(57) ABSTRACT

Compositions and methods of using thereof for the prevention of sexually transmitted diseases resulting from infection with one or more of viral pathogens have been developed. The compositions contain one or more porphyrins, tetrapyrrole macrocycle compounds with bridges of one carbon joining the pyrroles. In a preferred embodiment, the compositions are administered in a formulation suitable for administration to a mucosal surface.

### CROSS REFERENCE TO RELATED APPLICATIONS

Priority is claimed to U.S. Provisional application Ser. No. 60/347,197, filed Jan. 8, 2002.

### STATEMENT REGARDING FEDERALLY FUNDED RESEARCH

The Federal Government has certain rights in the invention disclosed herein by virtue of Grant No. AI45883 from the National Institute of Health to Richard W. Compans.

### BACKGROUND OF THE INVENTION

This application relates to the field of chemical compounds, specifically synthetic porphyrin compounds, for the prevention of sexually transmitted diseases (STDs) caused by pathogens such as human immunodeficiency virus and herpes viruses.

Sexually transmitted diseases (STDs), once called venereal diseases, are among the most common infectious diseases in the United States today. More than 20 STDs have now been identified, and they affect more than 13 million men and women in this country each year. The annual comprehensive cost of STDs in the United States is estimated to be well in excess of $10 billion.

STDs affect men and women of all backgrounds and economic levels. They are most prevalent among teenagers and young adults. Nearly two-thirds of all STDs occur in people younger than 25 years of age. The incidence of STDs is rising, in part because in the last few decades, young people have become sexually active earlier yet are marrying later. In addition, divorce is more common. The net result is that sexually active people today are more likely to have multiple sex partners during their lives and are potentially at risk for developing STDs.

Health problems caused by STDs tend to be more severe and more frequent for women than for men, in part because the frequency of asymptomatic infection means that many women do not seek care until serious problems have developed. Some STDs can spread into the uterus (womb) and fallopian tubes to cause pelvic inflammatory disease (PID), which in turn is a major cause of both infertility and ectopic (tubal) pregnancy. The latter can be fatal. STDs in women also may be associated with cervical cancer. One STD, human papillomavirus infection (HPV), causes genital warts and cervical and other genital cancers. STDs can be passed from a mother to her baby before, during, or immediately after birth; some of these infections of the newborn can be cured easily, but others may cause a baby to be permanently disabled or even die.

HIV Infection and AIDS

AIDS (acquired immunodeficiency syndrome) was first reported in the United States in 1981. It is caused by the human immunodeficiency virus (HIV), a virus that destroys the body's ability to fight off infection. An estimated 900,000 people in the United States are currently infected with HIV. People who have AIDS are very susceptible to many life-threatening diseases, called opportunistic infections, and to certain forms of cancer. Transmission of the virus primarily occurs during sexual activity and by sharing needles used to inject intravenous drugs.

Genital Herpes (HS)

Genital herpes affects an estimated 60 million Americans. Approximately 500,000 new cases of this incurable viral infection develop annually. Herpes infections are caused by herpes simplex virus (HSV). The major symptoms of herpes infection are painful blisters or open sores in the genital area. These may be preceded by a tingling or burning sensation in the legs, buttocks, or genital region. The herpes sores usually disappear within two to three weeks, but the virus remains in the body for life and the lesions may recur from time to time. Severe or frequently recurrent genital herpes is treated with one of several virucidal drugs that are available by prescription. These drugs help control the symptoms but do not eliminate the herpes virus from the body. Suppressive virucidal therapy can be used to prevent occurrences and perhaps transmission. Women who acquire genital herpes during pregnancy can transmit the virus to their babies. Untreated HSV infection in newborns can result in mental retardation and death.

Genital Warts

Genital warts (also called venereal warts or condylomata acuminata) are caused by human papillomavirus, a virus related to the virus that causes common skin warts. Genital warts usually first appear as small, hard painless bumps in the vaginal area, on the penis, or around the anus. If untreated, they may grow and develop a fleshy, cauliflower-like appearance. Genital warts infect an estimated 1 million Americans each year. In addition to genital warts, certain high-risk types of HPV cause cervical cancer and other genital cancers. Genital warts are treated with a topical drug (applied to the skin), by freezing, or if they recur, with injections of a type of interferon. If the warts are very large, they can be removed by surgery.

Other Sexually Transmitted Diseases

Other diseases that may be sexually transmitted include chlamydial infection, syphilis, Gonorrhea, trichomoniasis, bacterial vaginosis, cytomegalovirus infections, scabies, and pubic lice. STDs in pregnant women are associated with a number of adverse outcomes, including spontaneous abortion and infection in the newborn. Low birth weight and prematurity appear to be associated with STDs, including chlamydial infection and trichomoniasis. Congenital or perinatal infection (infection that occurs around the time of birth) occurs in 30 to 70 percent of infants born to infected mothers, and complications may include pneumonia, eye infections, and permanent neurologic damage.

HIV and AIDS

AIDS, or acquired immunodeficiency disease, is characterized by an imbalance in two basic types of immune system cells, helper/inducer T lymphocytes and suppressor T lymphocytes, with the ratio of suppressor cells to helper/inducer cells greatly elevated. Helper/inducer T cells, defined by a surface antigen called CD4, are responsible for the induction of most of the functions of the human immune system, including the humoral immune response involving the production of antibodies by B lymphocytes and the cell-mediated response involving stimulation of cytotoxic T cells. A condition associated with HIV is AIDS-related complex, or ARC. Most patients suffering from ARC eventually develop AIDS.

Two related retroviruses can cause AIDS, human immunodeficiency virus type 1 and type 2 (HIV-1 and HIV-2, generally referred to herein as HIV). The genomes of the two viruses are about 50% homologous at the nucleotide level, contain the same complement of genes, and appear to attack and kill the same human cells by the same mechanism. Also known as LAV (lymphadenopathy-associated virus), HTLV-3 (human T-lymphotropic virus-type 3), and ARV (AIDS-related virus), HIV-1 was identified in 1983. Virtually all AIDS cases in the U.S. are associated with HIV-1 infection. HIV-2 was isolated in 1986 from West African AIDS patients.

Both types of HIV are retroviruses, in which the genetic material is RNA rather than DNA. The viruses carry with them a polymerase (reverse transcriptase) that catalyzes transcription of viral RNA into double-helical DNA. The viral DNA can exist as an unintegrated form in the infected cell or be integrated into the genome of the host cell. As presently understood, the HIV enters the T4 lyphocyte where it loses its outer envelope, releasing viral RNA and reverse transcriptase. The reverse transcriptase catalyzes synthesis of a complementary DNA strand from the viral RNA template. The DNA helix then inserts into the host genome where it is known as the provirus. The integrated DNA may persist as a latent infection characterized by little or no production of virus or helper/inducer cell death for an indefinite period of time. When it is transcribed by the infected lymphocyte, new viral RNA and proteins are produced to form new viruses that bud from the cell membrane and infect other cells.

No treatment capable of preventing or reversing the immunodeficiency of AIDS or ARC is currently available. All patients with opportunistic infections and approximately half of all patients with Kaposi's sarcoma die within two years of diagnosis. Attempts at reviving the immune systems in patients with AIDS have been unsuccessful.

A number of compounds have apparent virucidal activity against this virus, including HPA-23, interferons, ribavirin, phosphonoformate, ansamycin, suramin, imuthiol, penicillamine, carbovir, 3′-azido-3′-deoxythymidine (AZT, and other 2′,3′-dideoxynucleosides, such as 2′,3′-dideoxycytidine (DDC), 2′,3′-dideoxyadenosine (DDA), 2′,3′-dideoxyinosine (DDI), 3′-azido-2′,3′-dideoxyuridine (CS-87), 2′,3′-dideoxy-2′,3′-didehydrocytidine (D4C), 3′-deoxy-2′,3′-didehydrothymidine (D4T) and 3′-azido-5-ethyl-2′,3′-dideoxyuridine (CS-85). However, all are administered systemically, are expensive, and have serious side effects. The virus also readily mutates to yield drug resistant strains. Systematic use of porphyrin compositions as antiviral drugs is described by U.S. Pat. Nos. 5,109,016 and 5,192,788 to Dixon, et al., but these compounds have not been tested clinically. U.S. Pat. Nos. 5,109,016 and 5,192,788 do not describe the use of porphyrin compounds as virucidal drugs which prevent initial viral infections.

Inhibitors of cellular processes will often limit viral replication. Unfortunately, they are also usually toxic for the host and therefore cannot be prescribed for a prolonged period of time because of their toxicity. Efforts to decrease the problem of toxicity have primarily been directed towards finding selective, less toxic drugs. Due to the exorbitant cost of the nucleoside type drugs, research has also been centered around compounds which are relatively easy and economical to manufacture.

Herpes Simplex

Another class of common STD viral pathogens are herpes simplex viruses, for example, herpes simplex virus type 2 (HSV-2). Following transmission of the virus to a susceptible individual, HSV-2 replicates in the epithelial cells of genital mucosal surfaces. This replication is usually asymptomatic, as evidenced by the number of individuals who are seropositive for HSV-2 antibody, but have no history of symptomatic infection. However, particularly in individuals who are seronegative for both HSV-1 and HSV-2, primary infection can result in severe, ulcerative lesions. Following replication in epithelia, the virus infects the peripheral endings of sensory neurons innervating the site of infection, and is transported through the neuronal axons to the nuclei. Viral DNA enters the neuronal nuclei and latent infections are established. Various stimuli, including stress, damage to peripheral tissues near the site of infection, or direct nerve damage cause reactivation of latent virus, and productive viral replication is initiated in the neuron. Virus is transported back through neuronal axons to the epithelial tissue, where it again replicates, is shed into extracellular space, and is available for transmission to a new individual.

Because the latent infection lasts for the lifetime of the host, infection by HSV has the potential to result in many episodes of recurrent disease and transmission. As with the initial infection, many of these recurrent infections are asymptomatic, so that neither the infected individual or his or her partner may be aware of the risk of transmission. Regular use of virucidal compounds by women who believe they are uninfected would reduce not only their own risk of infection, but would reduce the risk of transmission to new partners of women with asymptomatic recurrences.

In addition to genital infection, HSV-2 is the most common cause of neonatal herpes infection, which are most frequently transmitted during delivery of an infant to a mother who is shedding infectious virus (Whitley, et al. Ann Intern Med. 125(5):376-83 (1996)). Availability of nontoxic, topical virucidal compounds, and their use during delivery, would reduce or eliminate virus available for transmission and thereby also reduce the level of risk to the infant.

Genital herpes infections have also been implicated in the transmission of human immunodeficiency viruses. Epidemiologic studies have suggested that infection by HSV-2, along with other sexually transmitted diseases that cause genital ulcers, increases the risk of acquisition of HIV. The mechanism of this increased risk in unknown, but it may be due to the increased numbers of HIV-susceptible cells (CD4+ T cells and macrophages) present in genital epithelium during the inflammatory immune response generated by the STDs (Latif et al., AIDS. 3:519-523 (1989). In addition, co-infection of HSV-2 and HIV may result in a higher risk of transmission of HIV: HIV virions have been detected in cells present in genital lesions caused by HSV, leading to the hypothesis that HSV lesions may generated a higher level of HIV in the genital tract available for transmission.

There are a number of virucidal drugs available for inhibition of HSV replication, including acyclovir, cidofivir, sorivudine, and foscarnet. However, all of these drugs target replication of the viral DNA following infection of susceptible cells; they cannot prevent the initial infection of epithelial cells. In animal models, several of the drugs have been shown to be only partially effective at reducing viral replication in genital epithelium when applied topically (see, for example, Bravo, et. al., Antiviral Res 21:59-72 (1993)). Reduction of epithelial replication during initial infection has been demonstrated in animal models to reduce the amount of latent virus present in ganglia and to reduce the frequency and severity of recurrent disease (Roizman and Sears, Annu. Rev. Micrbiol. 1987, Vol. 41: 543-571 (1987)). However, other studies have demonstrated that epithelial replication is not a prerequisite for the establishment of latent infection in animal models (Sedarati et al., Virology 192:687-691 (1993)). In addition, the high percentage of women with latent virus but no history of symptomatic infection suggests that in humans, high levels of replication may not be necessary for the establishment of latency. In the absence of an effective vaccine, use of topical virucidal agents may then be the best chance for reducing the number of individuals with latent HSV-2 infections.

Anti-HSV virucides tested to date include compounds with both specific and nonspecific activity. Many of these compounds are effective virucides when tested in cell culture, including those that inhibit specific interactions between the virus and the cell surface (neutralizing antibodies and polyanionic compounds such as heparan sulfate, heparin, dextran sulfate, and carageenan), and those that disrupt virion architecture (nonoxynol-9) (see, for example, Zacharopoulos and Phillips, Clinical and Diagnostic Laboratory Immunology 4:465-468 (1997)). Polyanionic compounds have had varying success in inhibition of HSV-2 infection in vivo; in a mouse model of genital infection, heparan sulfate was not particularly effective, and dextran sulfate and carageenan prevented infection only of extremely low doses of virus (10³ pfu or less) (Zeitlin et al., Contraception 56: 329-335 (1997)). Continual use of nonoxynol-9 has been shown to cause inflammation of vaginal and cervical epithelium (See, for example, Stafford et al., Journal of AIDS and Human Retrovirology 17: 327-331 (1998)), and to inhibit growth of normal vaginal flora (lactobacilli) that protect the vaginal tract from infection by other pathogens (Stafford et al. 1998). Results of studies to determine the effects of N9 use on transmission of STDs, particularly HIV, have varied (Weir et al., Genitourin Med, 71:78-81 (1995)), but it seems far from an ideal topical microbicide for frequent vaginal application.

It is well documented that an active STD contributes to the increase in HIV transmission (Cohen, Science 279:1854-1855 (1998)). Successful treatment of STDs reduces genital shedding of HIV, thus lowering the HIV transmission rate (Cohen, 1998). Although two of the most common STDs, gonorrhea and chacroid, can be treated successfully, the development of antibiotic resistance may seriously compromise efforts to control these STDs. For example, high level of resistance to penicillin and tetracycline in H. ducreyi and N. gonorrhoeae has been recognized since 1976 (Ison et al., Antimicrobial Agents and Chemotherapy 42:2919-2922 (1998)). The percentage of resistant isolates in the New World to either penicillin or tetracycline approached 40% in 1995 (Ison et al., 1998). Although vaccine development against common STDs has a high priority and has been stimulated by the genome approaches, effective and safe vaccine against gonorrhea, syphilis and chlamydia are not yet in sight. Therefore, there is a need for a drug for the prevention of initial infection by HIV.

It is therefore an object of the present invention to provide compounds having virucidal activity against Human Immunodeficiency virus with little or no toxicity.

It is a still further object of the present invention to provide compounds having virucidal activity or for mucosal administration.

### SUMMARY OF THE INVENTION

Compositions for the prevention of STDs such as an infection caused by HIVs, HSVs, hepatitis B and C viruses, and papilloma viruses been developed. These contain one or more porphyrins or a pharmaceutically acceptable salt thereof. The porphyrins have one of the following structures: 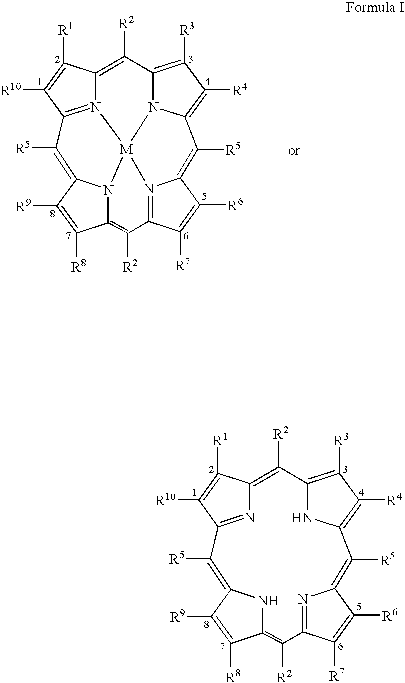
wherein R¹, R², R³, R⁴, R⁵ R⁶, R⁷, R⁸, R⁹, R¹⁰, R¹¹ and R¹² taken independently or together can be hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, halo, nitro, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, heteroarylthio, substituted heteroarylthio, cyano, isocyano, substituted isocyano, carbonyl, substituted carbonyl, carboxyl, substituted carboxyl, amino, substituted amino, amido, substituted amido, sulfinyl, substituted sulfinyl, sulfonyl, substituted sulfonyl, sulfonic acid, substituted sulfonic acid, phosphonato, substituted phosphonato, phosphoramide, polyaryl, substituted polyaryl, C1-C20 cyclic, substituted C1-C20 cyclic, heterocyclic, substituted heterocyclic, aminoacid, peptide, or polypeptide group, and
wherein M is a main group or transition metal atoms which optionally binds to one or more ligands. Representative metal atoms are gallium (Ga), aluminum (Al), cadmium (Cd), ruthenium (Ru), rhodium (Rh), platinum (Pt), osmium (Os), iridium (Ir), iron (Fe), cobalt (Co), zinc (Zn), molybdenum (Mo), titanium (Ti), manganese (Mn), chromium (Cr), nickel (Ni), magnesium (Mg), copper (Cu), indium (In), vanadium (V), silver (Ag), gold (Au), and tin (Sn).

Porphyrins are tetrapyrrole macrocycle compounds with bridges of one carbon joining the pyrroles. Many porphyrins are isolated from nature, for example, protoporphyrin. Many porphyrins are made synthetically, for example, those synthesized by condensation of aldehydes and pyrroles such as tetraphenylporphyrin. Derivatives of porphyrins include porphyrins with one or more substituents on one or more of the rings, porphyrins in which the conjugation of the ring has been altered by addition of substituents, porphyrins in which one or more center nitrogens is attached to substituents such as metals, liganded metals, and organic moieties, metalloporphyrins and metalloporphyrin-ligand complexes.

Effective concentrations for inactivation of the viral pathogens leading to STDs or HIVs vary with the STD, method of administration, severity of the disease and whether or not other drugs are being administered. Effective concentrations for inhibition of HIV-1, as measured in vitro by inhibition of replication range in PMB cells from 0.01 to greater than 100 μM.

The composition can be formulated in formulations suitable for any mode of administration. Preferred modes of administration are topical, or mucosal administration. In a specifically preferred embodiment, the mode of administration is administration via female genital tract or rectal administration, for a period of time effective to prevent infections.

### BRIEF DESCRIPTION OF THE DRAWINGS

FIGS. 1a-e. Structures of porphyrins studied: FIG. 1a, metalloporphyrins; FIG. 1b, TPPS4; FIG. 1c, sulfonated tetraaryl porphyrin; FIG. 1d, TNapPs; FIG. 1e, TAnthPS.

FIG. 2 is a graph of the activity of metalloTPPS4 against HIV-1 IIIB, measured as percent virus inactivated.

FIG. 3 is a graph of the activity of sulfonated tetraarylporphyrins against HIV-1 IIIB, measured as percent virus inactivated.

FIG. 4 is a graph of the concentration dependence of activity, measured as percent virus inactivated. HIV-1 IIIB virus samples were mixed with different concentrations of compounds (50 μg/ml, 5 μg/ml or 0.5 μg/ml), incubated in the dark for 1 hr, diluted 10-fold, and used to inoculate MAGI cells. Residual activity was determined as described for FIG. 2.

FIG. 5 is a graph of the kinetics of inactivation of HIV-1 IIIB. Compounds at a concentration of 50 μg/ml were mixed with virus and incubated at various time intervals: 0, 15, 30, 45, 60 minutes, diluted 1:10 with complete medium, and infectivity titers determined as described in FIG. 2.

FIG. 6 is a graph of the inhibition of gp120-CD4 binding by various porphyrins. A 96-well plate coated with soluble CD4 was incubated with HIV-1 IIIB gp120 in the presence or absence of compounds for 1 hr at room temperature. After extensive washes the bound gp120 was detected by anti-gp120 peroxidase-conjugated antibodies. Results represent % of gp120 binding compared to untreated gp120 samples (100%).

FIG. 7 is a graph of the the activity of various porphyrins against HIV-1 IIIB, HIV 1, SIVmac1A11, and A/PR/8/34, measured as percent virus inactivated.

### DETAILED DESCRIPTION OF INVENTION

Pharmaceutical porphyrin compositions for preventing sexually transmitted diseases (“STDs”) and the method of using the porphyrin compositions are provided herein. The pharmaceutical composition contains a synthetic porphyrin or a metalloporphyrin compound in an amount effective to inactivate a virus prior to an infection caused by the virus being effected. The composition may optionally include one or more pharmaceutically effective agents such as antibiotics, virucidals, antifungals, immunostimulants, and substances which are effective in inactivating viruses.

I. Definitions

The term “natural porphyrins” (NPs) as used herein refers to naturally occurring porphyrins or porphyrins synthesized de novo to resemble naturally occurring porphyrins.

The term “synthetic porphyrins” (SPs) as used herein refers to synthetic porphyrins or porphyrins derived synthetically from naturally occurring porphyrins.

The term “modified porphyrins” (MPs) as used herein refers to natural or synthetic porphyrins being modified by chemical reaction with one or more organic or inorganic groups including a metal or metal grouping. Therefore, the term “MNPs” refers to natural porphyrins modified with one or more organic or inorganic groups including a metal or metal grouping. The term “MSPs” refers to synthetic porphyrins modified with one or more organic or inorganic groups including a metal or metal grouping.

The term “metalloporphyrins” as used herein refers to any metal-porphyrin complexes. The metal can be any of the main group or transition metal atoms in one or more oxidation states. The metal can have one or more of various neutral ligands or negatively charged ligands. Metalloporphyrins may be in the form of a single molecule or aggregated molecules such as a dimer, a trimer, or tetramer.

II. Porphyrins

Porphyrins are tetrapyrrole macrocycle, compounds with bridges of one carbon joining the pyrroles. There are many different classes of porphyrins. Some porphyrins are isolated from nature and are termed natural porphyrins, for example, protoporphyrin IX, which is the organic portion of hemin. Many derivatives of natural porphyrins are known. Many porphyrins are synthesized in the laboratory. These include those made via the condensation of aldehydes and pyrroles, such as tetraphenylporphyrin. They also include porphyrins built up from smaller organic fragments. All porphyrins can have substituents off any of the positions of the ring periphery, including the pyrrole positions and the meso (bridging one carbon) positions as well as the central nitrogens. There can be one or more substituents, and combinations of one or more different substituents. The substituents can be symmetrically or unsymmetrically located.

The compositions disclosed herein contain one of more of porphyrins having the following structure: 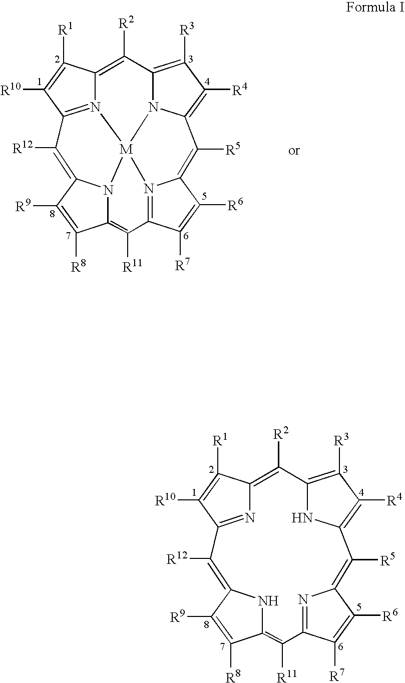
wherein R¹, R², R³, R⁴, R⁵, R⁶, R⁷, R⁸, R⁹, R¹⁰, R¹¹ and R¹² taken independently or together can be hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, halo, nitro, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, heteroarylthio, substituted heteroarylthio, cyano, isocyano, substituted isocyano, carbonyl, substituted carbonyl, carboxyl, substituted carboxyl, amino, substituted amino, amido, substituted amido, sulfinyl, substituted sulfinyl, sulfonyl, substituted sulfonyl, sulfonic acid, substituted sulfonic acid, phosphonato, substituted phosphonato, phosphoramide, polyaryl, substituted polyaryl, C1-C20 cyclic, substituted C1-C20 cyclic, heterocyclic, substituted heterocyclic, aminoacid, peptide, or polypeptide group,
wherein M is a metal atom selected from the group consisting of main group or transition metal atoms which optionally binds to one or more ligands.
   Representative metal atoms are gallium (Ga), aluminum (Al), cadmium (Cd), ruthenium (Ru), rhodium (Rh), platinum (Pt), osmium (Os), iridium (Ir), iron (Fe), cobalt (Co), zinc (Zn), molybdenum (Mo), titanium (Ti), manganese (Mn), chromium (Cr), nickel (Ni), magnesium (Mg), copper (Cu), indium (In), vanadium (V), silver (Ag), gold (Au), and tin (Sn);
or a pharmaceutically acceptable salt thereof.

The substituents, as well as the overall structure, of the porphyrins disclosed herein can be neutral, positively charged or negatively charged.

Charged structures have counterions, and many counterions and combinations of counterions are possible. Porphyrins can be covalently attached to other molecules, for example a cyclodextrin (Gonzalez, M. C.; Weedon, A. C. Can. J. Chem. 63, 602-608 (1985); Lang et al. Tetrahedron Lett. 43:4919-4922 (2002); Carofiglio et al. J. Org. Chem. 65:9013-9021 (2000); Weber et al. J. Chem. Soc. Chem. Commun. 1992:301-303 (1992)) or other sugar derivative (Schell et al. Bioorg. Med. Chem. 7:1857-1865 (1999); Sol et al. J. Org. Chem. 64:44314444 (1999); Csik et al. J.

Photochem. Photobiol. B. 44:216-224 (1998); Cornia et al., J. Org. Chem. 59:1226-1230 (1994)). They can have an attached molecular superstructure. The conjugation of the ring can be altered by addition of one or more substituents. One example would be a chlorin ring system (Vicente, M. G. H. In: Kadish, K. M.; Smith, K. M.; Guilard, R., Eds. The Porphyrin Handbook, San Diego: Academic Press; Vol. 1:149-199 (2000)). Another example would be a bacteriochlorin system (Sutton et al. Bioconjug. Chem. 13:249-263 (2002); Cavaleiro et al., J. Heterocycl. Chem. 37:527-534 (2000)).

Exemplary natural porphyrins of formula I are given below:

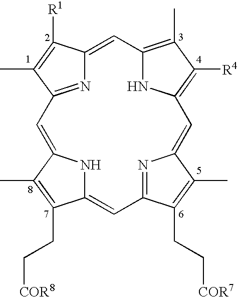

| Code | R¹, R⁴ | R⁷, R⁸ |
|---|---|---|
| NP-1 | CH(CH₃)OCH₂CH₂OCH₂CH₃ | OH |
| NP-2 | CH═CH₂ | OH |
| NP-3 | CH(CH₃)OH | OH |
| NP-4 | CH(CH₃)SCH₂CH₂-N[CH₂CH₃]₂O | OH |
| NP-5 | CH(CH₃)OBu | NHCH₂CO₂H |
| NP-6 | CH═CH₂ | Gly-OEt |
| NP-7 | CH(CH₃)OH | NHCH₂CO₂H |
| NP-8 | CH(CH₃)O(CH₂)₂NMe₂ | NHCH₂CO₂H |
| NP-9 | CH(CH₃)OCH₂CH₂OCH₂CH₃ | OCH₂CH₂OCH₂CH₃ |
| NP-10 | CH═CH₂ | OMe |
| NP-11 | CH₂CH₂OH | OMe |
| NP-12 | CH(CH₃)O(CH₂)₂NMe₂ | NMe₂ |
| NP-13 | CH(CH₃)OCH₂CH₂OCH₂CH₃ | NH(CH₂)₂N(CH₃)₂ |
| NP-14 | CH═CH₂ | NH(CH₂)₂N(CH₃)₂ |
| NP-15 | CH(CH₃)OH | NH(CH₂)₂N(CH₃)₂ |
| NP-16 | CH(CH₃)S(CH₂)₂N(CH₂CH₃)₂O | NH(CH₂)₂N(CH₃)₂ |

Exemplary synthetic porphyrins of formula I are given below. The structures of additional exemplary synthetic porphyrins are given below the table:

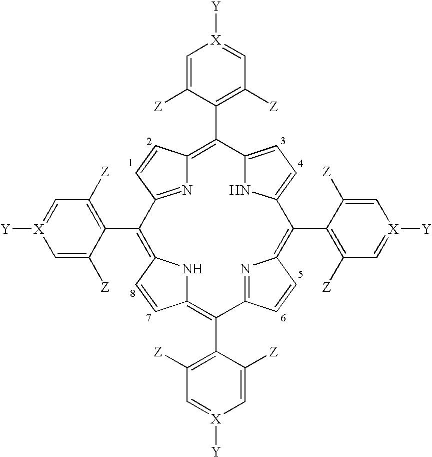

| Code | X | Y | Z |
|---|---|---|---|
| SP-1 | C | OH, or OR | H |
| SP-2 | C | SO₃H | H, or Cl |
| SP-3 | C | OCOR, or NHCOCH₂CH₂COOH | H |
| SP-4 | N | CH₃, or CH₂CH₂CH₂CH₃ | H |

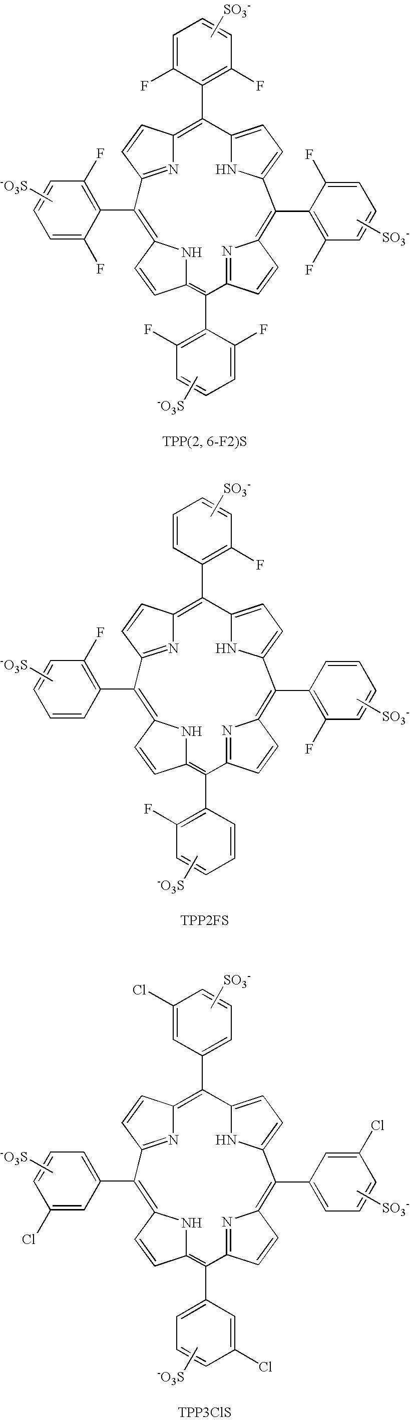

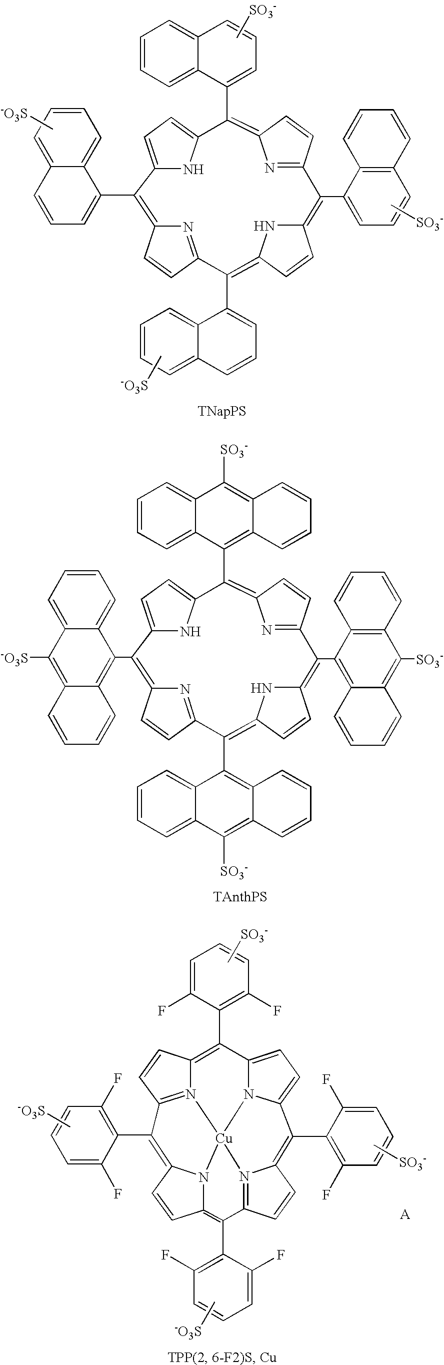

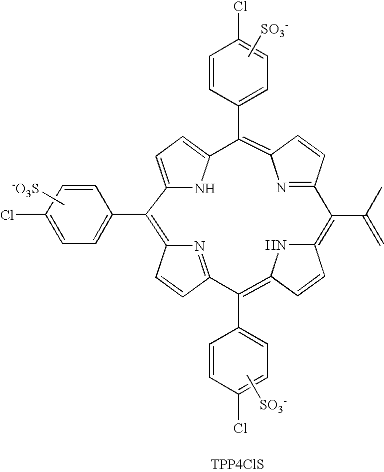

Other exemplary NPs, SPs, MNPs, and MSPs are described in the U.S. Pat. Nos. 5,281,616; 5,109,016; and 5,192,788, to Dixon et al. As used herein, except in combination with a carrier for application directly to the mucosal tissue, for example for the treatment or prevention of sexually transmitted disease, the porphyrin compounds defined in Formula I do not encompass the porphyrin compounds described in U.S. Pat. Nos. 5,109,016 and 5,192,788. In particular, U.S. Pat. Nos. 5,109,016 and 5,192,788 describe the following porphyrin compounds which were tested as effective for inhibition of HIV viruses and/or HSV viruses: 5,10-diphenyl-15,20-di(N-methyl-3-pyridyl)-porphyrin; 5,10-diphenyl-15,20di(N-methyl-4-pyridyl)-porphyrin; 5,15-diphenyl-10,20-di(N-methyl-3-pyridyl)-porphyrin; Cu(II)-5,10-diphenyl-15,20-di(N-methyl-4-pyridyl)-porphyrin (Cu-CP4); Ni(II)-5-10-diphenyl-15,20-di(N-methyl-4-pyridyl)-porphyrin (N-1-CP4); hemin; protoporphyrin; tetra-(N-methyl-4-pyridyl)-porphyrin; mesotetraphenylporphine; protoporphyrin IX dimethyl ester; tetra-(4-carboxyphenyl)-porphyrin; tetra(4-methylphenyl)-porphyrin; tetra-(3-methylphenyl)porphyrin; tetra-(4-hydroxyphenyl)-porphyrin; Fe(III)-tetraphenyl-porphyrin; tetra-(4-chlorophenyl)-porphyrin; Fe(III)-tetra-(4-methylphenyl)-porphyrin; Fe(III)-tetra-(N-methyl-4-pyridyl)-porphyrin; tetra-(N-methyl-4-pyridyl)-porphyrin tosylate salt; and Fe(III)-mu-oxo-dimer of tetraphenylporphyrin.

Representative metals include but are not limited to Ga, Al, Ga, Cd, Ru, Rh, Pt, Os, Ir, Fe, Co, Zn, Mo, Ti, Mn, Cr, Ni, Mg, Cu, Ti, In, Ru, V, Ag, Au, Sn. Additional ligands can be attached to the metal.

A variety of porphyrins have been found to have selective activity against HIV-1 and HIV-2 when tested in cell culture. Both natural and synthetic porphyrins and metalloporphyrins were tested for inhibition of reverse transcriptase. Compounds tested included, for example, 5,10-Diphenyl-15,20-di(N-methyl-3-pyridyl)-porphyrin; 5,10-Diphenyl-15,20-di(N-methyl-4-pyridyl)-porphyrin; 5,15-Diphenyl-10,20-di(N-methyl-3-pyridyl)-porphyrin; Hemin; Protoporphyrin; Tetra-(N-methyl-4-pyridyl)-porphyrin; Meso-tetraphenylporphine; Protoporphyrin IX dimethyl ester; Tetra-(4-carboxyphenyl)-porphyrin; Tetra-(4-methylphenyl)-porphyrin; Tetra-(3-methylphenyl)-porphyrin; Tetra-(4-hydroxyphenyl)-porphyrin; Fe(II)-tetraphenyl-porphyrin; Tetra-(4-chlorophenyl)-porphyrin; Fe(III) -tetra-(4-metbylphenyl)-porphyrin; Fe(lI)-tetra-(N-methyl-4-pyridyl)-porphyrin; and Fe(III)-μ-oxo-dimer of tetraphenylporphyrin. Additional compounds tested included TNapPS, sulfonated 5,10,15,20-tetra-naphthalen-1-yl-porphyrin; TAnthPS, sulfonated 5,10,15,20-tetra-anthracen-9-yl-porphyrin; TMPS, sulfonated tetramesitylporphyrin, sulfonated 4-chloroTPP (TPP4Cl,S); sulfonated 2-fluoroTPP (TPP2F,S); sulfonated 2,6-difluoroTPP [TPP(2,6-F2)S] and its copper chelate [TPP(2,6-F2)S,Cu].

A. Antiviral Properties of Porphyrins and Metalloporphyrins

There have been limited studies of porphyrins as antiviral species, i.e. those that prevent viral replication in already infected cells: Debnath et al. Med. Chem. Res. 9:267-275 (1999); Song et al., Antiviral Chem. Chemother. 8:85-97 (1997); Neurath et al., J. Mol. Recognition. 8:345-357 (1995); Debnath et al. J. Med. Chem. 37:1099-1108 (1994); Neurath et al.

Antiviral Chem. Chemother. 4:207-214 (1994); Feorino et al. Antiviral Chem. Chemother. 4:55-63 (1993); Ding et al. Biochem. Pharmacol. 44:1675-1679 (1992); Neurath et al. Antiviral Chem. Chemother. 2:303-312 (1991); Asanaka et al. AIDS. 3:403404 (1989). All of these reports are of the antiviral activities of the porphyrins. There are no reports of the virucidal activity of the porphyrins, i.e., of their preventing infection in previously uninfected cells. There are also reports of the use of light and porphyrins to kill viruses via photodynamic effects: Gabor et al. Photochem. Photobiol. 73:304-311 (2001); Stojiljkovic et al. Expert Opinion on Investigational Drugs. 10:309-320 (2001); North et al., J. Photochem. Photobiol. B. 17:99-108 (1993); Kasturi et al. Photochem. Photobiol. 56:427-429 (1992); North et al. Blood Cells. 18:129-140 (1992). These studies are not directly relevant because the invention claimed herein does not involve the use of light to activate the porphyrin or metalloporphyrin.

B. Porphyrin Hydrophobicity

There is documentation that the hydrophobic interactions of the planar extended aromatic porphyrin ring help stabilize its interactions with biomolecules (see, for example, Stephen J. Lippard and Jeremy M. Berg, Principles of Bioinorganic Chemistry, University Science Books, 1994). Other significant interactions include hydrogen bonding and electrostatic interactions of the peripheral substituents, and axial interactions involving the metal (Lippard & Berg, 1994). Another interaction mode, known for heme c, involves covalent linkages via thioether bonds derived from porphyrin vinyl groups and protein cysteine residues. Porphyrins can also have other “secondary” effects and interactions. These secondary types of interactions increase the number of possible modes of actions that must be considered in drug design.

The hydrophobicity of porphyrins disclosed herein can be evaluated by using, for example, capillary electrophoresis (see, for example, Bowser et al., Electrophoresis 18:82-91 (1997)). Therefore, by analyzing the hydrophobicities of various porphyrins disclosed herein, it is possible to establish the relationship between hydrophobicity and a particular porphyrin structure, thereby allowing the prediction of highest possible hydrophobic interactions of the porphyrin with a biomolecule.

C. Synthesis of Porphyrins

The porphyrins can be synthesized using general synthetic techniques. See, Dolphin, D. Ed., “The Porphyrins”, Vol. 6, Chap 3-10, pp. 290-339 (Academic Press: New York, 1979); Morgan, B., Dolphin, D. Struct. Bonding (Berlin), 64 (Met. Complexes Tetrapyrrole Ligands 1), pp. 115-203 (1987); Smith, Kevin M.; Cavaleiro, Jose A. S. Heterocycles, 26(7), 1947-63 (1987); Shanmugathasan et al., Tetrahedron. 56:1025-1046 (2000); Sternberg et al. Tetrahedron. 54:4151-4202 (1998); Sessler, J. L. In: Montanari, F.; Casella, L., Eds. Metalloporphyrins Catalyzed Oxidations Dordrecht: Kluwer Academic Publishers; 1993:49-86; Smith Adv. Exp. Med. Biol. 193:277-292 (1985); Inubushi et al. Methods Enzymol. 76:88-94 (1981). The chemistry related to porphyrins is well documented (see, e.g., The Porphyrin Handbook; Karl M. Kadish, Kevin M. Smith, Roger Guilard (eds.), Academic Press, San Diego, c2000, Volumes I to XV (1998)).

Still other synthetic techniques include the advances by Lindsey, et al., J. Org. Chem. 52, 827-836 (1987); Momenteau, M.; Loock, B.; Huel, C.; Lhoste, J. M. J. Chem. Soc., Perkin Trans. I, 283 (1988); Morgan, B.; Dolphin, D. J. Org. Chem. 52,5364-5374 (1987); Smith, K. M.; Parish, D. W.; Inouye, W. S. J. Org. Chem. 51, 666-671 (1986); and Smith, K. M.; Minnetian, O. M. J. Chem. Soc., Perkin Trans. 1, 277-280 (1986).

Other references to metal insertion include Buchler, J. E., “The Porphyrins”, vol. 1, ch. 10, Dolphin, D., ed. (Academic Press, N.Y. 1979); Lavallee, D. K. Coord. Chem. Rev. 61, 55-96 (1985); Lavallee, D. K. Comments Inorg. Chem. 5, 155-174 (1986); Hambright, P. In: Kadish, K. M.; Smith, K. M.; Guilard, R., Eds. The Porphyrin Handbook, Vol. 3. San Diego: Academic Press; 2000:129-210.

Porphyrins may also be obtained from commercial sources including Aldrich Chemical Co., Milwaukee, Wis., Frontier Scientific, Logan, Utah, and Midcentury Chemicals, Posen, Ill.

### Anionic and Cationic Synthetic Porphyrins (SPs)

These porphyrins can be synthesized according to methods and procedures documented and available in the art. Extensive synthetic routes are now available (Kadish and Smith, 2000). The SPs can be readily obtained by the classic Rothemund synthesis of TPP; this route involves the acid-catalyzed condensation of pyrrole with an aromatic aldehyde. Sulfonated porphyrins can be synthesized from sulfonated precursors: Nohr and Macdonald International Patent (WO99/36476), 1999. Beta-pyrrole sulfonated porphyrins can be synthesized: Garcia-Ortega et al., J. Porph. Phthalo. 4:564-568 (2000). Chlorosulfonation can be used: Rocha Gonsalves et al. Heterocycles. 43:829-838 (1996). The synthesis and separation of different isomers of anionic tetraphenylporphyrins (TPP) and their derivatives are well established (Srivastava et al., J. Org. Chem. 38:2103 (1973); Hambright, P. In: Kadish, K. M.; Smith, K. M.; Guilard, R., Eds. The Porphyrin Handbook, Vol. 3. San Diego: Academic Press; 2000:129-210; Sutter, et al., J. Chem. Soc. Faraday Trans. 89:495-502 (1993);). In another example, an Fe derivative of an octasulfonated porphyrin having two sulfonated and three methyl groups per phenyl ring was prepared (Song et al., Antivir. Chem. Chemother. 8:85-97 (1997)).

A second series of anionic TPP analogs is based on carboxylate acid derivatives. Carboxylate porphyrins are usually synthesized via a Rothmund condensation with a starting benzaldehyde bearing derivatived carboxylic acid groups. Alternatively, it is also possible to derivatize other functionalities to give a side chain ending in a carboxylic acid. For example, an anilino TPP derivative was functionalized with carboxylic acid derivative followed by loss of water to give the porphyrin dimer (Dixon et al., Antivir. Chem. Chemother. 3:279-282 (1992)).

Cationic tetraphenylporphyrins can be made which are, for example, derivatives of cationic TPPs based either on the pyridine or aniline structures, TPyP and TMAP (for example, Dixon et al., Ann. N.Y. Acad. Sci. 616:511-513 (1998)). The syntheses of these compounds are well documented (see, for example, Yue et al., Inorg. Chem. 30:3214 (1991); Dixon, et al., Antiviral Chemistry and Chemotherapy 3:279-282 (1992); Marzilli et al., J. Am. Chem. Soc. 114:7575-7577 (1992); Mukundan, et al., Inorg. Chem. 33:4676-4687 (1994); Mukundan, et al., Inorg. Chem. 34:3677-3687 (1995); Petho, et al., Chem. Soc. Chem. Commun. 1993:1547-1548 (1993) Hambright, P. In: Kadish, K. M.; Smith, K. M.; Guilard, R., Eds. The Porphyrin Handbook, Vol. 3. San Diego: Academic Press; 2000:129-210; Liu et al. Synthetic Communications. 30:2009-2017 (2000); Dancil et al., J. Heterocycl. Chem. 34:749-755 (1997); Li et al., Biochim. Biophys. Acta. 1354:252-260 (1997); Jin et al. Chem. Commun. 1939-1940 (1996); Almarsson et al., J. Am. Chem. Soc. 117:4524-4532 (1995); Casas et al., J. Org. Chem. 58:2913-2917 (1993); Pandey et al. Tetrahedron. 48:7591-7600 (1992); McCurry et al. Polyhdreon. 9:2527-2531 (1990).). The synthesis of unsymmetrical derivatives of this class of compounds has also been described (Ding et al., New J. Chem. 14:421-431 (1990); Peng et al., Can. J. Chem. 72:2447-2457 (1994).)

### Natural-Based Porphyrins (NPs)

Natural-based porphyrins can be modified using various organic synthetic method available in the art (Kadish and Smith, 2000). Extensive synthetic routes are now available (Inubushi & Yonetani, Methods Enzymol. 76:88-94 (1981); Nishino, et al., J. Org. Chem. 61:7534-7544 (1996); Sessler, “The synthesis of meso-substituted porphyrins” in Metalloporphyrin Catalyzed Oxidations (Montanari, F. and Casella, L. eds.), Kluwer Academic Publishers, Dordrecht, pp. 49-86 (1993); Smith & Cavaleiro, Heterocycles 26:1947-1963 (1987); Stemberg et al., Tetrahedron 54:41514202 (1998)). Many of the synthetic efforts with NPs involve manipulation of the PPIX chains of the porphyrins. For example, a NP bearing amides was prepared by the method common to peptide synthesis using one of the most versatile and useful peptide coupling agents, BOP reagent (benzotriazol-1-yloxy-tris(dimethylamino)phosphonium hexafluorophosphate) (Castro et al. Synthesis. 11:751-752 (1976).

### Porphyrins Conjugated to Other Molecules

Porphyrins, both natural and synthetic, can be conjugated to a wide variety of other molecules. Porphyrins conjugated to sugar and sugar derivatives, including cyclodextrins, were discussed above. Porphyrin-peptides can be synthesized by the methods described in the art (see, e.g., Chaloin et al., Bioconjug. Chem. 12:691-700 (2001); De Luca et al., Journal of Peptide Science. 7:386-394 (2001); Arai et al., J. Chem. Soc. Perkin Trans.2. 1381-1390 (2000); Solladie et al. Tetrahedron Lett. 41:6075-6078 (2000); Arai et al., J. Chem. Soc. Chem. Commun. 1503-1504 (1999); Matthews et al., New J. Chem. 23:1087-1096(1999); Pispisaet al., J. Phys. Chem. B. 103:8172-8179 (1999); De Luca et al. Letters in Peptide Science. 5:269-276 (1998); Geier et al. Tetrahedron Lett. 38:3821-3824 (1997)). Derivatives of NPs at the 2- and 4- or 6- and 7-positions can be readily made by the established methods by Sternberg et al. Tetrahedron. 54:4151-4202 (1998); Kahl et al., J. Org. Chem. 62:1875-1880 (1997).).

### Preparation of Pure Metalloporphyrins

Generally, pure metalloporphyrins can be prepared by mixing a appropriate metal salt with an appropriate porphyrin. To date, almost every metal has been incorporated into porphyrin through numerous procedures as described by Buchler, “Static Coordination Chemistry of Metalloporphyrins” in Porphyrins and Metalloporphyrins; K. M. Smith, Ed.; Elsevier, New York, Chapter 5, (1975); Buchler, “Synthesis and properties of metalloporphyrins” in The Porphyrins, Vol. 1, Dolphin, D., Ed.; Academic Press, New York, chapter 10 (1978); Buchler, Comments on Inorg. Chemi. 6:175-191 (1987); Buchler, et al., Fresenius J. Anal. Chem. 348:371-376 (1994). This general procedure is known to one skilled in the art of coordination chemistry. Syntheses of organic-soluble, metal-carbon σ-bonded porphyrins are also known to one in the art (See, e.g., Kadish, Kevin M. Smith, Roger Guilard, supra). For example, [Co(NH₃)₅CH₃]²⁺ has been used to transfer the methyl group to Co(III)TM_{py}P(4)⁵⁺ to form Me₂Co(III)TM_{py}(4)³⁺ (Kofod et al. Inorg. Chem. 36:2258-2266 (1997); Kofod Inorg. Chem. 34:2768-2770 (1995)). The metal atoms may have neutral or ionic ligands. Exemplary neutral ligands include H₂O, pyridine, imidazoles, NH₃, alkylamines, ethers, oxygen, amino acid or peptide esters, phosphines, and alcohol. Other neutral ligands commonly used in coordination chemistry may also used.

Exemplary ionic ligands can be negative charged ligands such as Cl⁻, NO₂⁻, CN⁻, RS⁻, terminal N-bound amino acids or peptides). In general, porphyrin complexes are more exchange labile than their counterparts with the same metal but with other ligands attached. Also, alkyl or aryl ligands can be used Kadish et al. Inorg. Chem. 37:2693-2700 (1998).

II. Selection of Porphyrins for Inhibition of Viral Pathogens

A. Tests of Virucidal Activities of Porphyrins

One can screen the porphyrin compositions for inactivation of viral pathogens such as HIVs or HSVs by various experimental techniques. In one embodiment, the technique involves the inhibition of viral replication in human peripheral blood mononuclear cells. The amount of virus produced is determined by measuring the quantity of virus-coded reverse transcriptase (an enzyme found in retroviruses) which that is present in the culture medium. Another technique involves measuring inhibition of purified reverse transcriptase in a cell free system.

B. Quantitative Structure Activity Relationship (QSAR) Analysis of Biological Activity

QSAR can be used to provide guidance for the selection of the most effective porphyrin compounds disclosed herein for the prevention of STDs caused by viral and/pathogens or AIDs caused by HIVs. QSAR has wide application in guiding the design of new pharmaceutical agents. Successful use of QSAR can substantially shorten the time needed to develop a new drug. The most detailed, relevant example of QSAR guidance in the design of new porphyrins and metalloporphyrins as virucidal or antibacterial agents is a study of porphyrin and metalloporphyrin anti-HIV-1 agents binding to the gp120 V3 loop sequence (Debnath et al., J. Med. Chem. 37:1099-1108 (1994)). Approximately 20 porphyrins were tested as anti-HIV agents including various NPs and porphyrins in the TPP carboxylic acid family. Debnath et al., used comparative molecular field analysis (CoMFA) for their QSAR.

Another approach is to derive molecular parameters from a number of sources and use these in a multiple linear regression to predict relative activity. Parameters might include the surface area, volume and polarizability of the porphyrin (the ChemPlus module in HyperChem, Hypercube, Inc.) as well as the dipole moment, LUMO and HOMO (and derived parameters) and net charge from the electrostatic potential (Gaussian).

III. Other Agents

The virucidal formulation may optionally include one or more pharmaceutically effective agents such as antibiotics, virucidals, antifungals, immunostimulants, and substances which are effective in inactivating viruses. In one embodiment, the pharmaceutically effective agents include synthetic or natural drugs, natural or synthetic polymers, and antibodies. In one embodiment, the agent can be a microbicidal polymer such as one of cyclodextrins, polyethylene hexamethylene biguanide, a seaweed polymer such as Carraguard, and antimicrobial peptide such as one of definsins. In another embodiment, the pharmaceutically effective agent can be a drug that inactivates one or more viruses.

IV. Pharmaceutically Acceptable Formulations

Some porphyrins are water soluble and may be administered in sterile water or physiological saline or phosphate buffered saline (PBS). Many porphyrins are not water soluble and are preferably administered in pharmaceutically acceptable non-aqueous carriers including oils and liposomes. Solubility of the porphyrins can be increased by techniques known to those skilled in the art including introducing hydroxyl groups and changing the counter ions.

There may also be included as part of the composition pharmaceutically compatible binding agents, and/or adjuvant materials. The active materials can also be mixed with other active materials including antibiotics, antifungals, other virucidals and immunostimulants which do not impair the desired action and/or supplement the desired action. Another preferred mode of administration of the porphyrin compositions described herein is mucosal administration. A specifically preferred mode of mucosal administration is administration via female genital tract. A preferred mode of mucosal administration is rectal administration. Suitable carriers include ointments, creams, gels, lotions, suppositories, nanoparticles, and polymeric formulations (microparticles, pellets, disks, or vaginal rings).

The active materials described herein can be administered by any route. Most preferably, the active materials described herein can be administered by, for example, topical administration, in liquid or solid form.

Various polymeric and/or non-polymeric materials can be used as adjuvants for enhancing mucoadhesiveness of the porphyrin composition disclosed herein. The polymeric material suitable as adjuvants can be natural or synthetic polymers. Representative natural polymers include, for example, starch, chitosan, collagen, sugar, gelatin, pectin, alginate, karya gum, methylcellulose, carboxymethylcellulose, methylethylcellulose, and hydroxypropylcellulose. Representative synthetic polymers include poly(acrylic acid), tragacanth, poly(methyl vinylether-co-maleic anhydride), poly(ethylene oxide), carbopol, poly(vinyl pyrrolidine), poly(ethylene glycol), poly(vinyl alcohol), poly(hydroxyethylmethylacrylate), and polycarbophil. Other bioadhesive materials available in the art of drug formulation can also be used (see, for example, Bioadhesion—Possibilities and Future Trends, Gurny and Junginger, eds., 1990).

Typical excipients include a binder such as microcrystalline cellulose, gum tragacanth or gelatin; starch or lactose, a disintegrating agent such as alginic acid, Primogel, and corn starch; a lubricant such as magnesium stearate or Sterotes; and a glidant such as colloidal silicon dioxide. When the dosage unit form is a capsule, it may contain, in addition to material of the above type, a liquid carrier such as a fatty oil. Other dosage unit forms may contain other various materials that modify the physical form of the dosage unit, for example, as coatings. Materials used in preparing these various compositions should be pharmaceutically pure and non-toxic in the amounts used.

The solutions or suspensions may also include the following components: a sterile diluent such as water, saline solution, fixed oils, polyethylene glycols, glycerine, propylene glycol or other synthetic solvents; antibacterial agents such as benzyl alcohol or methylparabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

Carriers that will protect the active compound against rapid elimination from the body, such as a controlled release formulation, including implants and microencapsulated delivery systems, can be formed of biodegradable, biocompatable polymers such as polyanhydrides, polyglycolic acid, collagen, and polyhydroxyacids such as polylactic acid. Methods for preparation of such formulations will be apparent to those skilled in the art. Liposomal suspensions (including liposomes targeted to infected cells with monoclonal antibodies to viral antigens) can also be used. Methods for encapsulation or incorporation of porphyrins into liposomes are described by Cozzani, I.; Jori, G.; Bertoloni, G.; Milanesi, C.; Sicuro, T. Chem. Biol. Interact. 53, 131-143 (1985) and by Jori, G.; Tomio, L.; Reddi, E.; Rossi, E. Br. J. Cancer 48, 307-309 (1983). These may also be prepared according to methods known to those skilled in the art, for example, as described in U.S. Pat. No. 4,522,811 (which is incorporated herein by reference in its entirety). Other methods for encapsulating porphyrins within liposomes and targeting areas of the body are described by Sicuro, T.; Scarcelli, V.; Vigna, M. F.; Cozzani, I. Med. Biol. Environ. 15(1), 67-70 (1987) and Jori, G.; Reddi, E.; Cozzani, I.; Tomio, L. Br. J. Cancer, 53(5), 615-21 (1986).

V. Methods of Prevention of STDs

Generally, the composition described can be used to prevent a viral infection by administering to a human being the composition that contains an effective amount of a porphyrin and/or metalloporphyrin compound that inactivates a virus prior to an infection caused by the viruses being effected. Optionally, a pharmaceutically effective amount of one or more of other agents can be used in combination with the porphyrin and/or metalloporphyin compound.

The porphyrins and/or metalloporphyrins have broad-spectrum anti-viral activities. The porphyrins can be formulated for administration to individuals in need of prevention of STDs. The formulations are preferably for local or regional delivery, for example, to the mucosa of the reproductive tract, or intestinal tract, but may also be formulated for systemic delivery. The formulation is designed to administer an amount of porphyrin effective to prevent infection of the STD. The time of administration is determined based on standard clinical criteria, determined using other antibiotic or virucidal formulations, clearance rates, and STD to be treated.

The compositions disclosed herein can be used to prevent STDs caused by viral pathogens. Exemplary viruses include HIV viruses, HSV viruses, hepatitis B and C viruses, and papilloma viruses. The pharmaceutically effective amount varies with the type of STD. Typically, an effective amount of the porphyrin compound is less than or equal to 10 μM in the presence of a pharmaceutically acceptable carrier or diluent. The compounds described herein are included in the pharmaceutically acceptable carrier or diluent in an amount sufficient to exert a therapeutically useful inhibitory effect in vivo without exhibiting adverse toxic effects on the user. It is to be noted that dosage values also vary with the specific severity of the disease condition to be alleviated. It is to be further understood that for any particular subject, specific dosage regimens should be adjusted to the individual need and the professional judgment of the person administering or supervising the administration of the aforesaid compositions.

Other agents which can be used in combination with the porphyrin and/or metalloporphyrin compound include antibiotics, virucidals, antifungals, immunostimulants, and substances which are effective in inactivating viruses. In one embodiment, the pharmaceutically effective agents include synthetic or natural drugs, natural or synthetic polymers, and antibodies. In one embodiment, the agent can be a microbicidal polymer such as one of cyclodextrins, polyethylene hexamethylene biguanide, a seaweed polymer such as Carraguard, and antimicrobial peptide such as one of definsins. In another embodiment, the pharmaceutically effective agent can be a drug that inactivates one or more viruses.

The present invention will be further understood by reference to the following non-limiting examples

### EXAMPLE 1

### Identification of Porphyrins with High Virucidal Activity for HIV-1

Materials and Methods

Porphyrins

Porphyrins were obtained from Frontier Scientific (Logan, Utah) or Mid-century Chemicals (Posen, Ill.). Porphyrin designations are as follows: PP, protoporphyrin IX; MP, mesoporphyrin IX; HP, hematoporphyrin IX; DP, deuteroporphyrin IX; DPSS, deuteroporphyrin IX 2,4-disulfonic acid; DPEG, deuteroporphyrin IX 2,4 bisethylene glycol; CoproI; coproporphyrin I; TPP, mesotetra(4-sulfonatophenyl)porphine; TPPS3, meso-tetraphenylporphyrin trisulfonate; TNapPS, sulfonated 5,10,15,20-tetra-naphthalen-1-yl-porphyrin; TAnthPS, sulfonated 5,10,15,20-tetra-anthracen-9-yl-porphyrin; TMPS, sulfonated tetramesitylporphyrin (2,4,6-trimethyl substitution on each phenyl ring). In all other instances, an “S” at the end of the name indicates that the parent porphyrin was sulfonated. In most cases, these are compounds with different numbers of sulfonates and/or different positions of the sulfonates on the ring. Additional natural porphyrins (NP) include NP 1,2,4-di-Br-DP,Fe; NP2, PP dipropanol; NP3, MP dipropanol; NP4, PP di-beta-Ala amide, Fe; NP5, MP dipropanol,Fe (the metal chelate of NP3). An additional synthetic porphyrin (SP), SP 1, is tri(4-sulfonatophenyl)-mono(4-pyridyl)porphyrin.

Cell Lines

The mouse NIH/3T3 and human HEp2 cell lines were obtained from the American Type Culture Collection (Manassas, Va.). The recombinant cell lines human MAGI, monkey sMAGI, mouse 3T3.T4, 3T3.T4, 3T3.T4.CCR5, 3T3.T4.CXCR4; and human T-cell lines CEMx174 and HUT78 were obtained through the AIDS Research and Reference Reagent Program, Division of AIDS (NIH) (Bethesda, Md.). The human 293T cell line was provided by S. L. Lydy (Emory University, Atlanta, Ga.). NIH/3T3, HEp2, 3T3.T4, 3T3.T4.CCR5,3T3.T4.CXCR4, MAGI, sMAGI, and 293T cells were maintained in Dulbecco's minimal essential medium (DMEM) supplemental with 10% fetal calf serum. Cell lines RUT78 and CEMx 174 were maintained in RPMI 1640 medium supplemented with 10% fetal calf serum.

Viruses and Plasmids

For construction of recombinant vaccinia viruses, plasmids pRB2 land vRB 12 were kindly provided by Drs. Bernard Moss (NIH) and David Steinhauer (National Institute for Medical Research, London, United Kingdom). The 3′SHIV-89.6 plasmid was obtained from J. Sodroski (Harvard Medical School, Boston, Mass.). Recombinant vaccinia viruses expressing full length (VV-239env) and truncated (VV-239T) SIV mac239 envelope proteins were previously described by Ritter et al., Virology 197:255-264 (1993), and Vvenyl expressing the BH10 envelope protein was described by Owens and Compans, J. Virol. 63:978-982 (1989). A recombinant vaccinia virus encoding a truncated Env protein of HIV-189.6was constructed as follows. The HIV-189.6 truncated env gene was obtained by polymerase chain reaction (PCR) amplification from the HIV-189.6 plasmid with the following primers: the 5′-primer introducing an EcoR I site 5′-GAGAAGAATTCAGTGGCAATGAGAGTGAAGG-3′ the 3′; the primer introducing an Nhe I site and a premature stop codon after the codon for amino acid (aa)17 in the cytoplasmic domain 5′CCTGTCGGCTAGCCTCGATCATGGGAGG AGGGTCTGAAACGATAATG. The PCR product was then digested by EcoR I and Nhe I and ligated into EcoR I and Nhe 1—predigested pRB21 as a donor plasmid for vaccinia recombination. The recombinant vaccinia virus was obtained by a plaque selection system using a recipient vaccinia virus vRB 12 described by Blasco and Moss, Gene 158:157-162 (1995). The plasmid pIIIenv3-1 encoding the envelope protein of the HXB2 station of HIV-1 was obtained from the AIDS Research and Reference Reagent Program, Division of AIDS (NIH). The Tat-responsive HIV-LTR in pIIIenv3-1 was used to promote expression of HXB2 rev and env. The helper plasmid pCMVtat was kindly provided by Steven Bartz (Fred Hutchinson Cancer Research Center, Seattle, Wash.). The plasmids expressing SIVmac239 full length Env pCMV239Env(FL) and truncated Env pCMV239Env(T) were described by Vzorov and Compans, Virology 221:22-33, (1996). Virus-infected H9/HTLV-III_{B}NIH 1983 cells were obtained from the AIDS Research and Reference Reagent Program, and the supernatant was used to infect HUT78 cells. HIV-1 IIIB virus was produced by continued passage of infected HUT78 cells and virus stock was prepared as described by Vzorov and Compans, J. Virol. 74:8219-8225 (2000). To prepare HIV-189.6 virus, 293T cells were transfected with p89.6 (from the AIDS Research and Reference Reagent Program). At 48 h post transfection, DMEM was removed and the cells were washed once in RPMI. Then 2×10 CEMx 174 cells were added to a plate in 5 ml of RPMI containing 10% fetal calf serum and cocultured overnight. The following day, CEMx174 cells were removed from virus producing 293T cells and placed in T-25 flasks for continued passage. SIVmac1A11 virus stock was described previously (Vzorov and Compans, 2000).

Monoclonal Antibodies, Antisera, and Proteins

SIM.2 and SIM.4 antibodies recognizing human CD4 and recombinant soluble human CD4 were provided by the AIDS Research and Reference Reagent Program (NIH). The recombinant IIB gp120 protein (baculovirus-expressed) was obtained from Intracel (Cambridge, Mass.). Anti-mouse immunoglobulin G peroxidase conjugate was obtained from Sigma (St. Louis, Mo.).

Screening of Porphyrins for Virucidal Activity

Porphyrin stock solutions were prepared at concentrations of 5 mg/ml, diluted 100-fold in growth medium, and mixed with virus stock. Samples were left in the dark at room temperature for 1 hr. For MAGI or sMAGI assays, 25 μl of virus/compound mixture was mixed with 225 ll of growth medium containing DEAE-Dextran (15 μg/ml) and 50 μl added to wells with confluent monolayers of MAGI or sMAGI cells (on a 96 well plate). At 2 hr postinfection, an additional 200 μl of complete DMEM was added. After three days virucidal activity was measured by removal of the media, fixation with 1% formaldehyde and 0.2% glutaraldehyde and staining with 5-bromo-4 chloro-3-indolyl-β-Dgalactophyranoside (X-gal). There were about 50 to 60 separate blue nuclei per well for the positive control. Scoring of blue nuclei in a 96-well format was greatly enhanced by using a planar lens (Olympus; x4) to visualize the entire well. For determining virus titers, RT (Roche), MAGI (Kimpton and Emerman, J. Virol. 66:2232-2239 (1992)), or sMAGI (Chackerian et al., Virology 213:386-394 (1995)) assays were used. Comparison of the numbers in blue cells in wells infected with untreated virus was used to determine residual viral infectivity (expressed as a percentage). Numerical data reported are the averages of three experiments, each run in duplicate.

Procedure for Removal of Unbound Porphyrin

Filtration was used to separate free compounds from the virus. Initial tests were performed on a large scale (without virus) so that the concentration of porphyrin could be measured spectroscopically (1601 spectrometer; Cary). Stock solutions of the porphyrin (5 mg/ml) were diluted 100-fold with medium. This solution was in turn diluted 50-fold with Dulbecco's phosphate-buffered saline (PBS). This solution (9 ml) was placed in a filtration apparatus (Centriplus YM-100; 100,000 MWCO; Millipore, Bedford, Mass.) and centrifuged. After three serial filtrations, the experimental concentration was compared to that expected on the basis of simple dilution calculations. For TPPC, with carboxylic acid groups on each of the porphyrin phenyl rings, three serial filtrations-dilutions left about a factor of two more porphyrin in solution than expected from simple dilution calculations. A similar experiment was run with TPPS4,Cu. This sulfonated porphyrin did not pass through the membrane as readily. In this case, the three serial filtrations-dilutions left about a factor of 35 more porphyrin than expected from simple dilution calculations.

In the corresponding biological experiments, 50 μl of the virus-compound mixture was mixed with 450 μl of PBS and loaded into a reservoir with a filter (Microcon YM-100; Millipor Corporation). The sample reservoir was placed into an Eppendorf tube and spun at 10,000 rpm for 3 min. To collect the sample, the reservoir was inverted into a new Eppendorf tube and spun again recovery spin). The volume of the sample after the recovery spin (about 50 μl) was readjusted to 500 μl with PBS, and the reservoir was spun with a new filter. The procedure was repeated a total of four times. Mathematically, this should have resulted in a 1,000-fold dilution of the porphyrin. From the control experiments, we conclude that the actual dilution was probably about 500-fold for nonsulfonated porphyrins. The final volume was adjusted up to 100 μl with PBS. To this was added 100 μl of 2×DMEM containing 20% fetal bovine serum and 30 μg of DEAE-dextran/ml; 50 μl of the resulting solution was added to the MAGI cells. Controls were tested similarly.

Gp120-CD4 Binding Assay

To investigate the possible effect of porphyrin compounds on binding of HIV-1 IIIB gp120 to CD4, a gp120 CD4 binding assay was developed. The assay was developed as a modification of a capture gp120 ELISA kit (Intracel Corporation). Briefly, a 96-well plate was coated with soluble CD4 and 0.5 μg of HIV-1 IIIB gp120 per well was incubated in the presence or absence of test compounds for 1 hr at room temperature. After four washes with buffer to remove unbound proteins, the bound gp120 was detected by anti-gp120 peroxidase-conjugated antibodies and quantitated by the protocol provided by the manufacturer.

CD4-Anti-CD4 Binding Assay

A CD4-anti-CD4 binding assay was developed as a modification of the capture gp120 ELISA assay (Intracel Corporation). First, a 96-well plate coated with soluble CD4 was incubated with mouse monoclonal anti-CD4 antibodies SIM.2 or SIM.4 at concentrations of about 600 ng/ml, in the presence or absence of test compounds (50 μg/ml or 5×10⁶ pmoles/well). As a positive control for blocking of binding, soluble CD4 (100 pmoles/well) was used. After 1 hr incubation at room temperature the plate was washed four times. For detection of the bound anti-gp120, anti-mouse peroxidase conjugated antibodies were used as described above.

Cell Fusion Assays

For cell fusion assays, three different expression systems were used: (i) a recombinant vaccinia virus expression system which is able to express high levels of Env, (ii) a plasmid expression system which is able to express Env proteins in the absence of other HIV proteins or vaccinia virus proteins, and (iii) cells persistently infected with HIV-1 IIB or HIV-189.6. For recombinant vaccinia viruses expressing HIV-1 Envor SIV proteins, HEp2 cells were infected with a m.o.i. (multiplicity of infection) of 5. After 24 hr cells were collected and counted, and about 2.5×10³ were added to 3T3CD4CXCR4 or 3T3CD45CCR5 cell monolayers in 96-well plates in 100 μL of medium in the presence or absence of the test compounds.

For the second assay, 293T cells were transfected by the calcium phosphate precipitation method with the plasmid pIIIenv3-1 expressing the HIV-1 Env protein (HXB2 Env) with a long terminal repeat promoter and cotransfected with a helper plasmid pCMVTAT at a ratio of 10:1; or with plasmids expressing simian immunodeficiency virus (SIV) Env proteins using a cytomegalovirus (CMV) promoter. After 48 hr cells were collected and cocultured with uninfected cells as in the previous assay.

As a third system, HUT78 cells persistently infected with HIV-1 IIIB or CEMx174 cells persistently infected with HIV-189.6 was used. The infected cells were counted and cocultured with uninfected cells as in the previous assays.

For all fusion assays, after 5 hr or 20 hr of cultivation, the level of cell fusion induced by the untreated recombinant virus-infected cells and the extend of fusion inhibition by the test compounds was evaluated by microscopic observation. Fusion activities were determined by counting the nuclei in syncytia and comparing the resulting number with the total number of nuclei.

Cytotoxicity Test

A standard trypan blue exclusion test (Strober, Trypan blue exclusion test of cell viability, p. A.3.3-A.3.4, in J. E. Coligan and A. M. Kruisbeek (ed.), Current protocols in immunology, Wiley-Greene, New York, N.Y., 1994) was used. Compounds at a concentration of 50 μg/ml in growth medium were added to a 96-well plate with MAGI cells. After 72 hr cells were detached by standard trypsin solution (0.25% trypsin-0.05% EDTA) and diluted 1:10 in growth medium. To test cell viability, 1 part of 0.4% trypan blue and 9 parts of diluted cells were mixed, incubated the mixture about 2 min at room temperature, and applied a drop of the trypan blue/cell mixture to a hemacytometer. Using a binocular microscope, the stained (nonviable) and unstained (viable) cells were then counted. The fraction of viable cells was calculated as the number of unstained cells in the wells treated with compound as a percentage of the number in control wells.

Therapeutic Indices

The 50% cytotoxic concentration (CC₅₀) was defined as the concentration of compounds that reduced the viability of cells by 50% (calculated from four different concentrations of porphyrin). The concentration achieving 50% protection was defined as the 50% effective concentration (EC₅₀). The selective index value was defined as the CC₅₀/EC₅₀ ratio.

Results

Anti-HIV Activity of Porphyrins.

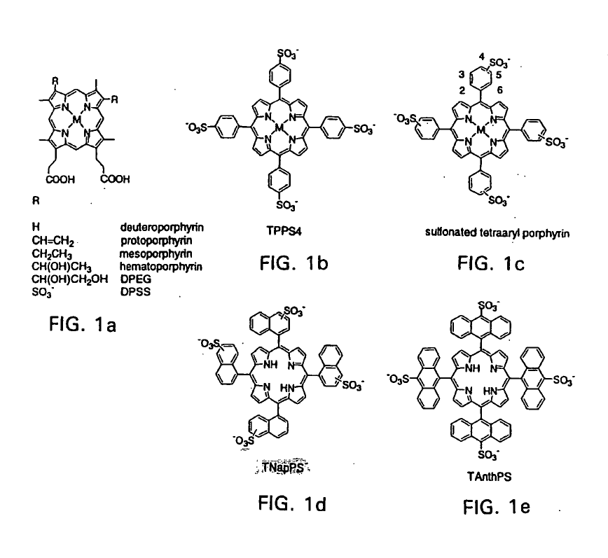

A series of natural and synthetic compounds (FIG. 1) were evaluated for their ability to inactivate the infectivity of HIV-1 IIIB virus using a MAGI cell assay. For structure-activity analysis, these porphyrins were divided into three classes: I) natural porphyrins; II) metallo-TPPS4 derivatives; and III) sulfonated tetraarylporphyrins. Each of these classes is discussed below.

Natural Porphyrins.

Initially, porphyrins related to protoporphyrin and its iron conjugate, hemin, were tested. The protoporphyrin ring skeleton has vinyl groups at the 2- and 4-position on the periphery of the ring (PP, Fe, Mn and Zn) (FIG. 1). Other related structures tested involved replacement of the vinyl groups on the heme periphery at the 2- and 4-positions: mesoporphyrin (MP; Cu and Mn), deuteroporphyrin (DP; Co, Cu, Fe, Mn and Zn), hematoporphyrin (HP; Co, Cu, Mn and Zn), the 2,4-bisethylene glycol derivative (DPEG; Fe and Zn), the 2,4-disulfonate (DPSS; Co, Cu, Fe and Zn and well as DPSSDME) and the 2,4-dibromo derivative (NP 1), protoporphyrin dipropanol (NP2) and mesoporphyrin dipropanol (NP3). NP2 and NP3 proved to be toxic. The tetracarboxylic acid Fe coproporphyrin I (Coprol,Fe) was tested as well. In general, only compounds with more than 80% inhibition of HIV growth under our assay conditions were studied in more detail. The natural porphyrins did not meet this criterion.

Some studies of porphyrin inhibition of viruses involve photoexcitation of a diamagnetic porphyrin, resulting in the production of singlet oxygen or free radicals or both which are the agents that damage the viruses (Matthews et al. Blood Cells 18:75-88 (1992); North et al. Photobiol. B. Biol. 17:99-108 (1993)). Photoactivation was not significant in the present study. In particular, diamagnetic derivatives (which are photoactive) were not in general more active than paramagnetic derivatives (which are not photoactive), e.g., the Fe(III) (paramagnetic), Mn(II) (paramagnetic) and Zn(II) (diamagnetic) derivatives of protoporphyrin gave 80, 65 and 52% inhibition, respectively, indicating that photoactivation does not play a significant role in viral inactivation.

Metallo-TPPS4 Derivatives.

A series of metallo derivatives of TPPS4 was evaluated (FIG. 1b). This series has the advantage that each porphyrin has a unique structure, e.g., that all sulfonates are in the 4-position and that each porphyrin has one (and only one) sulfonate on each of the phenyl rings. Metallo derivatives without axial ligands (TPPS4 and its Cu chelate, 93 and 97% inhibition, respectively) were more effective in preventing infection than derivatives with axial ligands (the Sn, Co and Gd chelates, 44, 63, and 68% inhibition, respectively). This relationship may indicate that axial ligands have undesirable steric interactions with the biological target. Some TPPS4 derivatives stack significantly in solution. To determine whether the monomeric form of the porphyrin was important for the activity, the self-stacking of these derivatives was evaluated by measuring the optical spectrum of each of the metalloTPPS4 derivatives as a function of added NaCl. This measurement gives data allowing a good estimate to be made of the relative ease of porphyrin stacking.

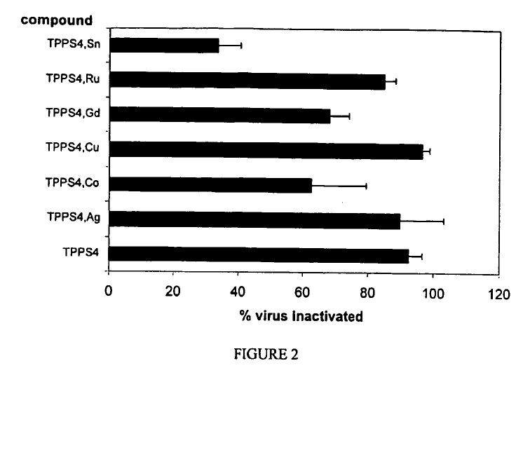

Porphyrins at a concentration of 50 μg/ml were incubated with HIV-1 IIIB in the dark for 1 hr, diluted 10-fold and used to inoculate MAGI cells. After three days activity against HIV was measured by removal of the media, fixation and staining with X-gal. The nuclei of infected cells were stained blue after incubation with X-gal. The residual HIV infectivity (%) was measured by dividing the number of blue cells in wells infected with compound-treated virus by the number in wells infected with untreated virus. The results are shown in FIG. 2. Data are reported as the mean of three independent assays, each run in duplicate. Error bars represent the standard deviation.

Self-stacking of the TPPS4 derivatives followed the order: TPPS4≅Ni≅Pd>Cu>VO>TiO>Ru, Mn. There was a general correlation between the propensity to self-stack in solution and the ability of these TPPS4 chelates to inhibit growth of HIV; the derivatives which self-stack were more active in blocking HIV infection. Because self-stacking is greater for derivatives without axial ligands (no metal, Cu, Ni), the effect may be due to enhanced binding of planar species at the biological site, rather than stacking per se. As observed for the natural porphyrins, there was no correlation between anti-HIV activity and the paramagnetic/diamagnetic nature of the central metal, indicating that photoactivation is not playing a role in virus inhibition.

Sulfonated Derivatives of TPP and Related Porphyrins.

These compounds are synthesized by sulfonation of the parent tetraaryl porphyrin (FIG. 1c). All are mixtures of compounds including members with different extents of sulfonation and perhaps different positions of the sulfonate on the ring (Sutter et al. J. Chem. Soc. Faraday Trans. 89:495-502 (1993)). Starting materials included TPP derivatives with 2-, 3- and 4-chloro substituents as well as the 2- and 4-fluoro substituents. More sterically hindered derivatives had 2,4,6-triMe, 2,6-diF and 2F, 5CF₃ substitution. The sulfonated naphthyl and anthracenyl porphyrins were also studied.

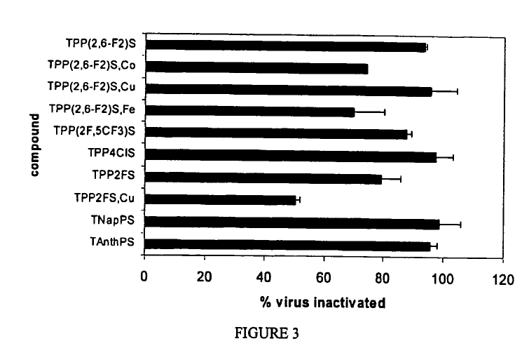

The activity of the sulfonated tetra-arylporphyrins against HIV-1 III was measured as described above with reference to FIG. 2. The results are shown in FIG. 3. Compounds giving greater than 80% inhibition of viral growth in initial screens were evaluated in more detail (FIG. 3). The five most active compounds were TNapS (FIG. 1d); TAnthPS (FIG. 1e); TPP(2,6-F2)S; TPP(2,6-F2)S,Cu; and TPP4ClS. All of these except the TPP4ClS have substantial steric bulk above and below the plane of the porphyrin. The results indicate that substitution above and below the plane of the porphyrin may enhance the activity of these species.

Effective Concentration.

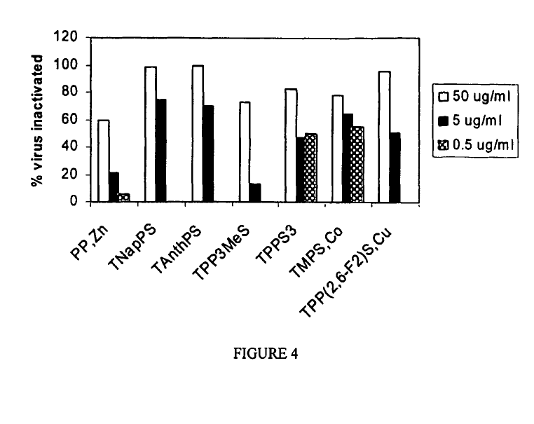

To determine the effective concentration of the compounds, virus samples were mixed with porphyrins at 10-fold dilutions of 50 μg/ml, 5 μg/ml, and 0.5 μg/ml. The most effective concentration was the highest concentration of 50 μg/ml (FIG. 4). However, three compounds also exhibited significant activity at concentrations of 0.5 μg/ml, specifically TNapPS, TAnthPS and TPP(2,6-F2)S,Cu. The most active compounds had an EC₅₀ of less than 5 μg/ml.

Kinetics of Inactivation.

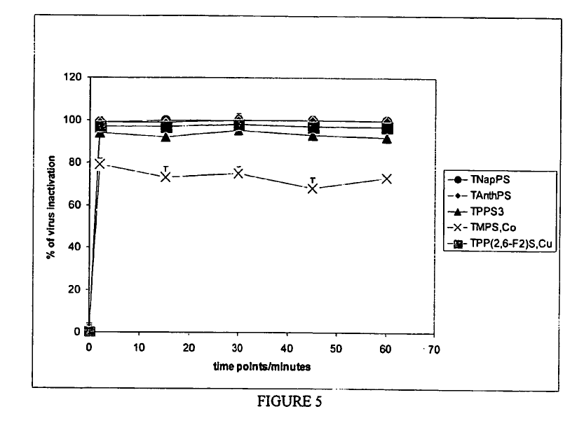

To determine the kinetics of inactivation of viral infectivity, mixtures of HIV-1 IIIB were incubated with five porphyrins at a concentration of 50 μl/ml and residual infectivity assayed at various time intervals (FIG. 5). For all these compounds, the activity observed at 2 minutes did not change over the time period studied (up to 60 minutes). This indicates that the interaction of these compounds with HIV-1 IIIB is very rapid and not time-dependent. TNapPS and TAnthPS inhibited viral growth almost completely in this assay. TPP(2,6-F2)S,Cu was only slightly less active. When these compounds were tested at a concentration of 5 μl/ml, similar levels of inactivation of virus was found at all time points, but generally the inactivation was less complete than at higher concentrations.

Virucidal Activity of Porphyrins.

A filtration-dilution method was used to determine whether the virus, once treated, was still rendered non-infectious once the unbound compound had been removed from the solution. Solutions of the virus and compound were filtered until only about 10% of the original volume remained. The solution that had not gone through the filter was diluted to the original volume and the process repeated four times. Spectroscopic assays showed that four dilutions resulted in the original porphyrin concentrations being reduced by 30- to 500-fold. For these filtration assays, compounds were selected in two categories: three active porphyrins [TNapPS, TAnthPS, and TPP(2,6-F2)S,Cu], and two porphyrins, with intermediate activity (TMPS,Co and TPP(2,6-F2)S,Fe).

TNapPS and TAnthPS had high anti-HIV activity in the screening assay (without removal of free compound) as well as after removal of compounds by the filtration-dilution method, with about 90-99% inactivation of the virus either with or without filtration. This demonstrates that the compounds exhibit virucidal activity; i.e., that the virus has been rendered noninfectious on the time scale of the experiment. TPP(2,6-F2)S,Cu inhibited about 95% of the virus in the screening assay and about 80% of the virus after filtration-dilution. TMPS,Co and TPP(2,6-F2)S,Fe had about 80% anti-HIV activity in the screening assay and about 20-40% anti-HIV activity after filtration-dilution. The partial recovery of virus infectivity observed with these compounds may be due to disassociation of the porphyrin from the viral envelope structure during the filtration-dilution.

Activity with Other Lentiviruses.

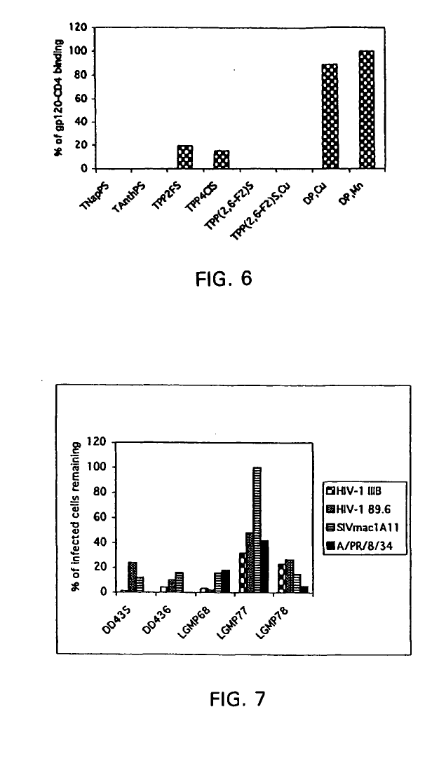

To investigate whether the compounds with high activity would inactivate other HIV strains and lentiviruses, the studies were extended to HIV-189.6 and SIVmac1A11. The results are shown in FIG. 6. The most active compounds against HIV-1 IIIB: TNapPS, TAnthPS and TPP(2,6-F2)S,Cu and two compounds with intermediate activity, TPPS4,Co and TPPS4,Ag, were tested. Both viruses were sensitive to the most active compounds: TNapPS, with 76% of 89.6 and 88% of SIVmac1A11 being inactivated; TAnthPS with 90% of 89.6 and 84% of 1A 11 being inactivated; and TPP(2,6-F2)S,Cu with 98% of HIV 89.6 and 84% of SIVmacSIVmac1A11 being inactivated. The compounds TPPS4,Co and TPPS4,Ag inactivated about 50-70% of HIV-189.6 infectivity. Thus, the porphyrins with activity against a laboratory-adapted virus (IIIB) were also active against a primary HIV isolate (89.6) as well as against SIV.

Toxicity.

A trypan blue exclusion test to determine possible toxicity of the test compounds. Compounds at a concentration of 50 μg/ml in growth medium were added to MAGI cells. This concentration is the same as that used for pretreatment of virus; however, it is ten-fold higher than that used when the compounds are applied to MAGI cells for virus assay. After 72 hr, a trypan blue assay was used to compare cell viability in cells treated with compounds to untreated cells. Of the three most active compounds, TAnthPS did not have any detectable toxic effect. TNapPS and TPP(2,6-F2)S,Cu showed 55% and 60% toxicity, respectively. The most active of the natural porphyrins, DPEG,Fe, also did not have any detectable toxic effect. Three natural porphyrins with no activity were also tested for toxicity. Cells treated with DP,Mn were 100% viable, with DP,Cu were about 71% viable, and with DP,Co were about 59% viable. TPP4ClS, the sulfonated TPP with one halogen with the best activity against HIV, showed about 50% toxicity. TPP3ClS, also a member of this class, was found to be too toxic for accurate measurement of activity of virus inhibition. The most active of the sulfonated TPP derivatives with two halogens, TPP(2,6-F2)S, showed about 50% toxicity. All of these data indicate that there is no correlation of virucidal activity and toxicity. A number of the most active compounds in each class showed no detectable toxic effect.

Therapeutic indices were measured for three of the most active compounds by measuring both activity and toxicity at four concentrations of porphyrin. The cytotoxic concentration (CC₅₀) was defined as the concentration that reduced the viability of cells by 50%; the effective concentration (EC₅₀) was defined as the concentration achieving 50% protection against HIV infection. The selective index value was defined as the CC₅₀/EC₅₀ ratio. TNapPS (DD435 EC₅₀=5 μg/ml; CC₅₀=75 μg/ml), TPP(2,6-F2)S,Cu (EC₅₀=5 μg/ml; CC₅₀=250 μg/ml), and TPPS3 (EC₅₀=5 μg/ml; CC₅₀=50 μg/ml) had CC₅₀/EC₅₀ values of 15, 50 and 10, respectively.

Effect of Porphyrins on Interaction of gp120 with CD4.

Effects on binding of gp120 to its primary receptor, CD4 have been investigated. CD4 binding results in a conformational change in gp120 that enables it to interact with a coreceptor, generally either CCR5 or CXCR4. To investigate the effect of porphyrins on binding of gp120 to CD4, a gp120-CD4 binding assay was used. The inhibition of binding using three groups of compounds was tested. Four of the porphyrins [TNapPS, TAnthPS, TPP(2,6-F2)S and TPP(2,6-F2)S,Cu] with highest activity against HIV were found to completely inhibit binding of gp120 to CD4 (FIG. 7). TPP4ClS showed about 97% inhibition of HIV and 85% inhibition of gp120/CD4 binding. TPP2FS had about 80% activity against HIV and 81% inhibition of gp120/CD4 binding. A third control group of porphyrins that did not have significant anti-HIV activity (e.g., DP,Cu and DP,Mn) also did not inhibit binding, or had only low activity.

A greater effect upon binding than infectivity was observed using the gp120-CD4 binding assay to investigate the effective concentration. These results show a general correlation between activity against HIV and inhibition of gp120 binding to CD4, although the latter was found to be more sensitive to inhibition by compounds with intermediate levels of activity against HIV.

Inhibition of HIV-Induced Cell Fusion by Porphyrins.

To determine if porphyrins bad an effect on the functional activity of the Env protein, the effects of the porphyrins were tested using assays for cell fusion activity (Table 1).

Three different expression systems were used for the Env proteins, which differ with respect to expression of other encoded proteins. Initially, a recombinant vaccinia expression system, which is able to express high levels of Env, was used. Experiments were run using a recombinant expressing the IIIB Env of HIV 1 which has tropism for the X4 coreceptor (VVenyl) and a recombinant expressing the 89.6 Env, a primary viral isolate with dual tropism for both X4 and R5 coreceptors (VV89.6 envt). Complete inhibition was observed of HIV-induced cell fusion with TNapPS, TAnthPS, and TPP(2,6-F2)S,Cu, which had excellent activity against HIV and completely blocked gp120/CD4 binding. Complete inhibition of fusion in all three assays in cells treated with TPPS4,Cu was also observed. This compound had intermediate levels of activity against HIV in the MAGI assay and blocked gp120/CD4 binding about 80-100%. Inhibition by these compounds of fusion induced by VVenyl was more extensive than that induced by VV89.6 envt (Table 1).

TABLE 1
Inhibition of Env-induced cell fusion by prophyrins^{a}

| | Presence of nuclei in syncytia at | | | |
|---|---|---|---|---|
| | indicated time(s) for construct: | | | |
| | pIIIenv | VVenvl | VV89.6 envt | VV89.6 envt |
| Compound | X4, 6 h | X4, 6/23 h | X4, 6/23 h | R5, 6/23 h |
| None | 1+ | 4+/4+ | 4+/4+ | 4+/4+ |
| TAnthS | − | −/− | −/− | −/− |
| TNaPS | − | −/− | −/− | −/− |
| DP,Cu | 1+ | 4+/4+ | 4+/4+ | 4+/4+ |
| DP,Mn | 1+ | 4+/4+ | 4+/4+ | 4+/4+ |
| DP,Co | 1+ | 4+/4+ | 4+/4+ | 4+/4+ |
| DP,Sn | 1+ | 4+/4+ | 4+/4+ | 4+/4+ |
| TPP2FS | − | −/4+ | 1+/4+ | 4+/4+ |
| TPP(2,6-F2)S | − | −/− | 1+/4+ | 1+/4+ |
| TPP(2,6-F2)S,Cu | − | −/− | −/− | −/− |
| TPP(2F,5CF3)S | − | −/4+ | 2+/4+ | 3+/4+ |
| TPPS4 | − | −/− | −/4+ | 1+/4+ |
| TPPS4,Cu | − | −/− | −/− | −/− |
| TPPS4,Ru | 1+ | −/− | −/4+ | 1+/4+ |
| TPPS4,Pd | − | −/− | −/4+ | −/4+ |
| TPPS4,Ni | 1+ | −/− | −/3+ | −/1+ |
| TPPS4,Mn | − | 2+/4+ | 4+/4+ | 4+/4+ |
| TPPS4,TiO | − | −/− | −/4+ | −/4+ |
| NP1 | − | 4+/4+ | 4+/4+ | 3+/4+ |
| NP2 | 1+ | 4+/4+ | 4+/4+ | 4+/4+ |
| HP,Co | 1+ | 4+/4+ | 4+/4+ | 4+/4+ |
| HP,Cu | − | −/− | 1+/4+ | 1+/2+ |
| HP,Zn^{b} | − | −/− | 1+/− | −/− |
| TPP3MeS^{b} | − | −/− | 1+/− | 2+/− |
| TPPS3 | − | −/− | 1+/4+ | −/4+ |

^{a}Fusion activities were demonstrated by comparing the nuclei in syncytia to the total nuclei. 4+, more than 50% of nuclei are in syncytia; 3+, 30 to 50% of nuclei are in syncytia; 2+, 30 to 10% of nuclei are in syncytia; + less than 10% of nuclei are in syncytia; −, no syncytia were observed.
^{b}Cells show toxicity at 23 h.

A plasmid expression system that is able to express Env proteins in the absence of other HIV proteins or vaccinia proteins (pIIIenv) was also used. As with the systems above, complete inhibition of HIV-induced cell fusion with compounds TNapPS, TAnthPS, or TPP(2,6-F2)S,Cu, were used. Many other compounds also exhibited complete inhibition in this assay.

Finally, fusion activity in cells persistently infected with HIV-1 IIIB or HIV-189.6 viruses was examined, which were cocultivated with uninfected target cells in the presence or absence of test compounds. The fusion activity observed in this assay was comparable with that found using plasmids expressing Env, and lower than observed with Env expressed by vaccinia virus. The results of fusion inhibition by the compounds tested correlated well with those observed using both other expression systems.

These results demonstrate that the porphyrins with high or intermediate levels of activity against HIV are able to effectively inhibit the membrane fusion activity of the viral Env proteins, a biological function that is important for viral entry as well as the induction of viral cytopathic effects.

Discussion

The central goal of these studies was to identify porphyrins with activity against HIV that could be useful as topical microbicides to provide a defense against sexual transmission of the virus. The vaginal and gastrointestinal surfaces play a major role in the pathogenesis of infection by HIV-1 as potential routes for viral entry. A MAGI assay was used to determine activity against HIV of test compounds that is based on usage of an epithelial cell line. Based on kinetics, effective concentration, and fusion inhibition, the most active compounds were TNapPS, TAnthPS, and TPP(2,6-F2)S,Cu. These compounds were also able to inhibit infection by dual tropic HIV-189.6 as well as SIVmac1A11 viruses. TNapPS and TAnthPS gave only approximately 1% infected cells remaining after 2 min incubation, indicating a very rapid inactivation.

A major mechanism for activity against HIV may involve porphyrin binding to the V3 loop of gp120. The results indicate that the porphyrins blocked binding of gp120 to CD4, and inhibited cell fusion activity of Env proteins when expressed from recombinant vectors. These results showed that an important target of these compounds is the viral Env protein. Neurath et al. (Neurath et al. 1992; Neurath et al. 1995) have correlated the anti-HIV activity using an assay for cytotoxicity in a T cell line, with the inhibition of interaction between gp120 and antibodies specific for the V3 hypervariable loop of this protein. In this series of approximately 20 porphyrins from both the natural and synthetic classes, there was no clear correlation overall between inhibition of antibody binding to gp120 and overall activity in an antiviral assay. However, there was a correlation in the most active members of the series. Debnath et al. have found an excellent correlation between predicted and observed anti-HIV-1 activity using a 3D-QSAR model (Debnath et al. 1994). For a data set composed primarily of natural porphyrins and TPPC derivatives, they observed that the active site apparently is best accommodated by a porphyrin bearing three negatively charged substituents and groups which can provide positive van der Waals interactions at positions corresponding to the 2- and 4-positions of protoporphyrin.

Porphyrins were able to inhibit the cell fusion activity of the HIV Env protein. To exclude the possibility that such an inhibitory effect could be due to an indirect effect, it was observed that cell fusion induced by recombinant vectors in the absence of any other HIV protein was also sensitive to inhibition by porphyrins. These results provide strong evidence that the porphyrins are able to effectively inhibit an important function of the Env protein that is needed for viral entry. Song et al. have also correlated anti-HIV activity with syncytium inhibition for a series of synthetic anionic porphyrins and metalloporphyrins (Song et al. 1997). No clear overall correlation was seen, but compounds with EC₅₀ vs. HIV of <10 μg/ml all had EC₅₀ values for syncytium inhibition of <40 μg/ml.

Currently several categories of compounds are undergoing thorough testing as potential microbicides to prevent HIV transmission. The first agents to be tested extensively were surface disruptive agents (surfactants, detergents) that kill or inactivate viruses (vaginal virucides) such as nonoxynol-9 (N9). Unfortunately, this class of compounds causes damage to human tissues, leading to inflammation and ulceration (Stafford et al. 1998). After extensive testing it was also determined that the use of this surfactant actually increases the risk of acquiring HIV infection during sexual transmission (Fichorova et al. 2001; Richardson et al. 2001; van de Wijgert and Coggins 2002) and its development as an agent to prevent HIV infection has therefore been discontinued. A second group of compounds includes peptides and antibodies, which enhance the normal vaginal defense mechanisms (Mascola 2002; Weber et al. 2001). A possible limitation of such compounds is the difficulty of their formulation for use as vaginal microbicides. A third group includes nonspecific enhancers of normal vaginal defense mechanisms (lactobacilli, acid buffers, peroxidases) (Clarke et al. 2002). These compounds did not fully inactivate individual virus particles that are potentially capable of infection at sites of injury. A fourth group includes polymers such as Carraguard (Spieler 2002). This vaginal microbicide gel containing the red seaweed extract, carrageenan, has been shown to block HIV and other sexually transmitted agents in vitro. However, such polymers may not be fully protective because of possible escape of some virus particles from interaction with the macromolecules. The sulfonated porphyrins are polyanionic molecules. They inhibit viral binding and fusion/entry into susceptible cells, as do some other polyanionic species, including polymers (De Clercq 2002). Porphyrins, however, are relatively small molecules and are convenient for formulation into vaginal gels. Their interaction with the virus appears to be very rapid. For some of the molecules studied, removal of free compound did not result in significant recovery of infectivity, indicating that they are effective virucidal agents. For other molecules, removal of free compound does result in partial recovery of infectivity, which possibly results from their dissociation from target sites on surfaces of virions. However, this is not an important concern for their use as microbicides, because the compounds will continue to be present at sites of transmission during exposure to virus in vivo.

These results demonstrate that the porphyrins with high or intermediate levels of activity against HIV are able to effectively inhibit the membrane fusion activity of the viral Env proteins, a biological function that is important for viral entry as well as the induction of viral cytopathic effects.

### EXAMPLE 2

### Identification of Porphyrins with Virucidal Activity for HSV-1 and HSV-2.

Porphyrin were Tested for Inactivation of HSV.

Materials and Methods

In vitro Assay

Either HSV-1(F) or HSV-2(G) (107 pfu) was mixed with 1 ml of porphyrin at the concentration indicated in Dulbecco's modified Eagle's medium (DME) with 1% newborn calf serum. All assays were performed in duplicate. All tubes were wrapped in foil to keep out light, and ambient room light was reduced as much as was practicable. Virus and porphyrin were incubated together at room temperature for times up to 60 minutes (Table 2). Following incubation, 10 fold serial dilutions were performed in DME with 1% newborn serum, using tubes wrapped in foil. Virus was diluted 20,000 fold and plated on Vero cells. Following a 2 hour incubation with the cells, the inoculum was removed and cells were overlaid with DME with 1% newborn calf serum and 0.2% human gamma globulin. Cells were fixed and stained, and plaques counted, 2 days after infection.

Infection Assay

5 week old female CBA/J mice were injected with Depo-Provera (2 mg/mouse) 5 days before infection. Gels (with drug or control) were 2% methylcellulose in PBS. 100 μl of gel was inserted into each mouse vagina, followed at various times by HSV-2(G) (105 pfu in 25 μl of DME with 1% newborn calf serum). 48 hours after infection, 25 μl of DME with 1% newborn calf serum was inserted into the mouse vagina, pipetted in and out several times, and collected for virus titration.

Results

Exemplary porphyrins and metalloporphyrins identified as active for inactivating HSVs include:
DPIX,Fe; HPIX,Fe; HPIX,Zn; PPIX,In; MPIX,Co; PPIX,Co; PPIX,Fe; PPIX,In; DPIX 2,4-bis ethylene glycol,Cu;
tetrakis(2,6-difluorosulfonatonatophenyl)porphyrin;
tetrakis(2,6-difluorosulfonatonatophenyl)porphyrin,Cu;
tetrakis(2,6dichlorosulfonatonatophenyl)porphyrin;
tetrakis(2-chlorosulfonatophenyl)porphyrin;
tetrakis(3-chlorosulfonatophenyl)porphyrin;
tetrakis(2-fluorosulfonatonatophenyl)porphyrin;
tetrakis(2-fluorosulfonatonatophenyl)porphyrin,Cu; TMesPS,Co;
TMesPS,Fe; TPPC4; TPPS3; TPPS3,Ag; TPPS3,Cu; TPPS3,Fe; TPPS3,Zn;
TPPS4,Ag; TPPS4,Cu; TPPS4,Fe; and TPPS4,Zn.

As used herein, DPIX is deuteroporphyrin IX; HPIX is hematoporphyrin IX; PPIX is protoporphyrin IX; MPIX is mesoporphyrin IX; TMesP is tetramesitylporphyrin; and TPPS3 is (5-phenyl-10,15,20-trisulfonatophenyl)porphine. Other exemplary porphyrins identified as active for inactivating HSVs include the sulfonated derivatives of tetrakis(1-naphthyl)porphyrin and tetrakis(2-naphthyl)porphyrin, including the parent porphyrin, and the corresponding Zn, Fe, and Cu chelates.

Those skilled in the art will recognize, or be able to ascertain using no more than routine experimentation, many equivalents to the specific embodiments of the invention described herein. Such equivalents are intended to be encompassed by the following claims.

1. A method for preventing a viral infection in a human comprising administering to a mucosal surface of a composition comprising a synthetic porphyrin or a pharmaceutically acceptable salt thereof to a human, wherein the porphyrin has the following structure: 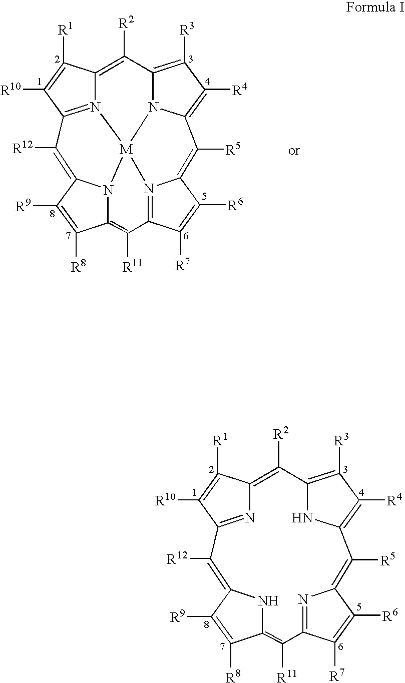
wherein R¹, R², R³, R⁴, R⁵, R⁶, R⁷, R⁸, R⁹, R¹⁰, R¹¹ and R¹² taken independently or together can be hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, halo, nitro, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, heteroarylthio, substituted heteroarylthio, cyano, isocyano, substituted isocyano, carbonyl, substituted carbonyl, carboxyl, substituted carboxyl, amino, substituted amino, amido, substituted amido, sulfinyl, substituted sulfinyl, sulfonyl, substituted sulfonyl, sulfonic acid, substituted sulfonic acid, phosphonato, substituted phosphonato, phosphoramide, polyaryl, substituted polyaryl, C1-C20 cyclic, substituted C1-C20 cyclic, heterocyclic, substituted heterocyclic, aminoacid, peptide, or polypeptide group, and
wherein M is a metal atom selected from the group consisting of main group or transition metal atoms which optionally binds to one or more ligands, and
wherein the porphyrin or the pharmaceutically salt thereof is in an effective amount to prevent the viral infection.

2. The method of claim 1 wherein the viral infection is caused by a virus selected from the group consisting of HIV viruses, HSV viruses, hepatitis B and C viruses, or papilloma viruses

3. The method of claim 1 wherein the viral infection is caused by a virus selected from the group consisting of HIV viruses.

4. The method of claim 1 wherein the viral infection is caused by a virus selected from the group consisting of HSV viruses.

5. The method of claim 1 wherein the viral infection is caused by a virus selected from the group consisting hepatitis B and C viruses, or papilloma viruses.

6. The method of claim 1 wherein the infection is caused by a virus selected from the group consisting of HIV and HSV viruses,
wherein R¹, R³, R⁴, R⁶, R⁷, R⁹, R¹⁰, and R¹² are hydrogen, and R², R⁵, R⁸, and R¹¹ are alkyl, heteroalkyl, aryl, or heteroaryl groups, and
wherein the porphyrin as a whole bears one or more sulfonic acid or derivatized sulfonic acid groups.

7. The method of claim 6 wherein the porphyrin is a Cu or Fe chelate of the structure of Formula 1.

8. The method of claim 1 where the infection is caused by a virus selected from the group consisting of HSV viruses,
wherein R², R⁵, R⁸, and R¹¹ are hydrogens and R¹, R³, R⁴, R⁶, R⁷, R⁹, R¹⁰, and R¹² are hydrogen, alkyl, heteroalkyl, or substituted alkyl groups, and
wherein the molecule as a whole bears two or more carboxylic acid groups.

9. The method of claim 8 wherein the porphyrin is a Cu or Fe chelate of the structure of Formula 1.

10. The method of claim 1 wherein the viral infection is caused by a virus selected from the group consisting of HIV viruses, and
wherein the compound is selected from the group consisting of TNapPS, TPP(2,6-F2)S,Cu; TAnthPS, TMPS,Co, DPEG,Fe, DPEG,Zn; TPPC,Fe; TPPC; TPP(2,6-C12)S,Fe and TPP(2,6C12)S; TPP2FS;TPP4ClS; TPP(2,6-C12); TPP(2,6-F2)S,Cu; TPP(2F,FCF3)S; and mixtures thereof.

11. The method of claim 1 where the viral infection is caused by a virus selected from the group consisting of HSV viruses, and
wherein the compound is selected from the group consisting of DPIX,Fe; HPIX,Fe; HPIX,Zn; PPIX,In; MPIX,Co; PPIX,Co; PPIX,Fe; PPIX,In; DPIX 2,4-bis ethylene glycol,Cu; tetrakis(2,6-difluorosulfonatonatophenyl)porphyrin; tetrakis(2,6-difluorosulfonatonatophenyl)porphyrin,Cu; tetrakis(2,6-dichlorosulfonatonatophenyl)porphyrin; tetrakis(2-chlorosulfonatophenyl)porphyrin; tetrakis(3-chlorosulfonatophenyl)porphyrin; tetrakis(2-fluorosulfonatonatophenyl)porphyrin; tetrakis(2-fluorosulfonatonatophenyl)porphyrin,Cu; TMesPS,Co; TMesPS,Fe; TPPC4; TPPS3; TPPS3,Ag; TPPS3,Cu; TPPS3,Fe; TPPS3,Zn; TPPS4,Ag; TPPS4,Cu; TPPS4,Fe; TPPS4,Zn; and the sulfonated derivatives of tetrakis(1-naphthyl)porphyrin and tetrakis(2-naphthyl)porphyrin, the Zn, Fe, and Cu chelates thereof, and mixtures thereof.

12. The method of claim 1 wherein the compound is protected against rapid elimination from the body.

13. The method of claim 1 further comprising providing the compound in a pharmaceutically acceptable carrier selected from the group consisting of ointments, creams, gels, lotions, troches, suppositories, vaginal rings, liposomes, nanoparticulates, microspheres, and controlled release formulations.

14. The method of claim 1 further comprising administering a therapeutically effective amount of at least one compound selected from the group consisting of antibiotics, virucidals, antifungals, and immunostimulants.

15. The method of claim 1 further comprising administering a therapeutically effective amount of at least one microbicide selected from the group consisting of carraguard, antibodies, defensins, cyclodextrins, polyethylene hexamethylene biguanide, and other compounds which are active in preventing viral infection.

16. The method of claim 14 wherein the virucidal is selected from the group consisting of HPA-23, interferons, ribavirin, phosphonoformate, ansamycin, suramin, imuthiol, penicillamine, carbovir, 3′-azido-3′-deoxythymidine (AZT), 2′,3′-dideoxycytidine (DDC), 2′,3′-dideoxyinosine (DDI), 2′,3′-dideoxyadenosine (DDA), 3′-azido-2′,3′-dideoxyuridine (CS-87), 2′,3′-dideoxy-2′,3′-didehydrocytidine (D4C), 3′-deoxy-2′,3′-didehydrothymidine (D4T) and 3′-azido-5-ethyl-2′,3′-dideoxyuridine (CS-85).

17. The method of claim 1 wherein the composition is administered topically or to the mucosa.

18. The method of claim 17 wherein the composition is administered to the female genital tract.

19. The method of claim 17 wherein the composition is administered rectally.

20. The method of claim 1 wherein the M is selected from the group consisting of gallium (Ga), aluminum (Al), cadmium (Cd), ruthenium (Ru), rhodium (Rh), platinum (Pt), osmium (Os), iridium (Ir), iron (Fe), cobalt (Co), zinc (Zn), molybdenum (Mo), titanium (Ti), manganese (Mn), chromium (Cr), nickel (Ni), magnesium (Mg), copper (Cu), indium (In), vanadium (V), silver (Ag), gold (Au), and tin (Sn).

21. The method of claim 20 wherein M is Cu.

22. The method of claim 20 wherein M is Fe.

23. A composition for mucosal administration for preventing a viral infection comprising a synthetic porphyrin or pharmaceutically active salt thereof having the following structure: 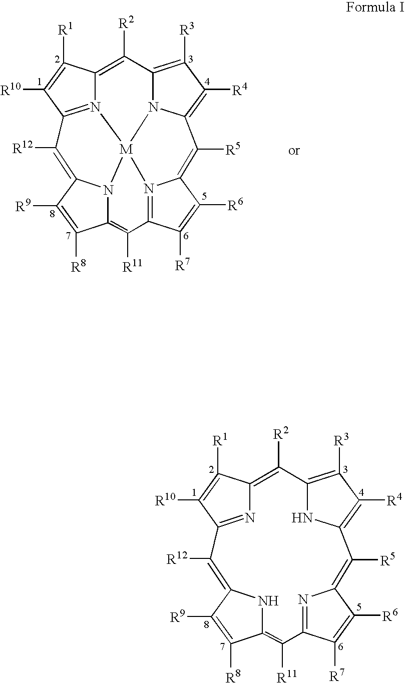
wherein R¹, R², R³, R⁴, R⁵, R⁶, R⁷, R⁸, R⁹, R¹⁰, R¹¹ and R¹² taken independently or together can be hydrogen, alkyl, substituted alkyl, alkenyl, substituted alkenyl, alkynyl, substituted alkynyl, phenyl, substituted phenyl, aryl, substituted aryl, heteroaryl, substituted heteroaryl, halo, nitro, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, heteroarylthio, substituted heteroarylthio, cyano, isocyano, substituted isocyano, carbonyl, substituted carbonyl, carboxyl, substituted carboxyl, amino, substituted amino, amido, substituted amido, sulfinyl, substituted sulfinyl, sulfonyl, substituted sulfonyl, sulfonic acid, substituted sulfonic acid, phosphonato, substituted phosphonato, phosphoramide, polyaryl, substituted polyaryl, C1-C20 cyclic, substituted C1-C20 cyclic, heterocyclic, substituted heterocyclic, aminoacid, peptide, or polypeptide group, and
wherein M is a metal atom selected from the group consisting of main group or transition metal atoms which optionally binds to one or more ligands, and
a pharamaceutically acceptable carrier for mucosal administration.
wherein the porphyrin or the pharmaceutically salt thereof is in an effective amount to prevent the viral infection.

24. The composition of claim 23 wherein the pharmaceutically acceptable carrier is selected from the group consisting of ointments, creams, gels, lotions, troches, suppositories, vaginal rings, liposomes, nanoparticulates, microspheres, and controlled release formulations.

25. The composition of claim 23 further comprising a therapeutically effective amount of at least one compound selected from the group consisting of antibiotics, virucidals, antifungals, and immunostimulants.

26. The composition of claim 23 comprising a therapeutically effective amount of at least one microbicide selected from the group consisting of carraguard, antibodies, defensins, cyclodextrins, and polyethylene hexamethylene biguanide.

27. The composition of claim 25 wherein the virucidal is selected from the group consisting of HPA-23, interferons, ribavirin, phosphonoformate, ansamycin, suramin, imuthiol, penicillamine, carbovir, 3′-azido-3′-deoxythymidine (AZT), 2′,3′-dideoxycytidine (DDC), 2′,3′-dideoxyinosine (DDI), 2′,3′-dideoxyadenosine (DDA), 3′-azido-2′,3′-dideoxyuridine (CS-87), 2′,3′-dideoxy-2′,3′-didehydrocytidine (D4C), 3′-deoxy-2′,3′-didehydrothymidine (D4T) and 3′-azido-5-ethyl-2′,3′-dideoxyuridine (CS-85).

28. The composition of claim 23 in a formulation for topical administration.

29. The composition of claim 23 in a formulation for administration via the female genital tract.

30. The composition of claim 23 wherein the porphyrin composition is effective for treating a sexually transmitted disease.

31. The composition of claim 23 in a formulation for administration via the rectum.

32. The composition of claim 23 wherein M is selected from the group consisting of gallium (Ga), aluminum (Al), cadmium (Cd), ruthenium (Ru), rhodium (Rh), platinum (Pt), osmium (Os), iridium (Ir), iron (Fe), cobalt (Co), zinc (Zn), molybdenum (Mo), titanium (Ti), manganese (Mn), chromium (Cr), nickel (Ni), magnesium (Mg), copper (Cu), indium (In), vanadium (V), silver (Ag), gold (Au), and tin (Sn).

33. The composition of claim 32 wherein M is selected from the group consisting of Cu and Fe.

34. The composition of claim 23 selected from the group consisting of TNapPS, TPP(2,6-F2)S,Cu; TAnthPS, TMPS,Co, DPEG,Fe, DPEG,Zn; TPPC,Fe; TPPC; TPP(2,6-C12)S,Fe and TPP(2,6-C12)S; TPP2FS; S; TPP(2,6-C12); TPP4ClS; TPP(2,6-F2)S,Cu; TPP(2F,FCF3)S; and mixtures thereof.

35. The composition of claim 23 wherein the composition is effective to inhibit infection or replication of a virus selected from the group consisting of HIV viruses and HSV viruses.

36. The composition of claim 23 wherein the composition is effective to inhibit infection or replication of a virus selected from the group consisting hepatitis B and C viruses, and papilloma viruses.

37. The composition of claim 35
wherein R¹, R³, R⁴, R⁶, R⁷, R⁹, R¹⁰, and R¹² are hydrogen, and R², R⁵, R⁸, and R¹¹ are alkyl, heteroalkyl, aryl, or heteroaryl groups, and
wherein the porphyrin as a whole bears one or more sulfonic acid or derivatized sulfonic acid groups.

38. The composition of claim 37 wherein the porphyrin is a Cu or Fe chelate of the structure of Formula 1.

39. The composition of claim 23 where the composition is effective to inhibit infection or replication of a HSV virus,
wherein R², R⁵, R⁸, and R¹¹ are hydrogens and R¹, R³, R⁴, R⁶, R⁷, R⁹, R¹⁰, and R¹² are hydrogen, alkyl, heteroalkyl, or substituted alkyl groups, and
wherein the molecule as a whole bears two or more carboxylic acid groups.

40. The composition of claim 39 wherein the porphyrin is a Cu or Fe chelate of the structure of Formula I.

41. The composition of claim 23 wherein the composition is effective to inhibit infection or replication of a HIV virus,
wherein the compound is selected from the group consisting of TNapPS, TPP(2,6-F2)S,Cu; TAnthPS, TMPS,Co, DPEG,Fe, DPEG,Zn; TPPC,Fe; TPPC; TPP(2,6-C12)S,Fe and TPP(2,6-C12)S; TPP2FS;TPP4ClS; TPP(2,6-C12); TPP(2,6-F2)S,Cu; TPP(2F,FCF3)S; and mixtures thereof.

42. The composition of claim 39 wherein the compound is selected from the group consisting of DPIX,Fe; HPIX,Fe; HPIX,Zn; PPIX,In; MPIX,Co; PPIX,Co; PPIX,Fe; PPIX,In; DPIX 2,4-bis ethylene glycol,Cu; tetrakis(2,6-difluorosulfonatonatophenyl)porphyrin; tetrakis(2,6-difluorosulfonatonatophenyl)porphyrin,Cu; tetrakis(2,6-dichlorosulfonatonatophenyl)porphyrin; tetrakis(2-chlorosulfonatophenyl)porphyrin; tetrakis(3-chlorosulfonatophenyl)porphyrin; tetrakis(2-fluorosulfonatonatophenyl)porphyrin; tetrakis(2-fluorosulfonatonatophenyl)porphyrin,Cu; TMesPS,Co; TMesPS,Fe; TPPC4; TPPS3; TPPS3,Ag; TPPS3,Cu; TPPS3,Fe; TPPS3,Zn; TPPS4,Ag; TPPS4,Cu; TPPS4,Fe; TPPS4,Zn; and the sulfonated derivatives of tetrakis(1-naphthyl)porphyrin and tetrakis(2-naphthyl)porphyrin, the Zn, Fe, and Cu chelates thereof, and mixtures thereof.

43. The composition of claim 23 wherein the porphyrin or a metal chelate of the porphyrin is covalently linked to one or more sugars or sugar derivatives.

44. The composition of claim 23 wherein the porphyrin or a metal chelate of the porphyrin is covalently linked to one or more amino acids or peptides.
